(19) United States
(12) Patent Application Publication
(10) Pub. No.: US 2012/0225131 A1
(43) Pub. Date: Sep. 6, 2012

(54) Methods For Treating Type Diabetes With Brown Adipose Transplants

(75) Inventors: DAVID W. PISTON, Nashville, TN (US); SUBHADRA C. GUNAWARDANA, Whites Creek, TN (US)

(73) Assignee: VANDERBILT UNIVERSITY, Nashville, TN (US)

(21) Appl. No.: 13/406,194

(22) Filed: Feb. 27, 2012

Related U.S. Application Data
(60) Provisional application No. 61/446,805, filed on Feb. 25, 2011.

(51) Int. Cl. A61K 35/12; A61P 3/10

(52) U.S. Cl. 424/574

(57) ABSTRACT

The present invention provides for methods of treating diabetes by providing to a subject brown adipose tissue. The tissue may be transplanted from a culture of the subject's own tissue (autograft) or from a donor. These methods can achieve normoglycemia and suppress hypergluconemia, and alleviate conditions associated with such, even in the absence of or at extremely low levels of adjunct insulin therapy, and without any appreciable increase in insulinogenesis.

This application claims benefit of priority to U.S. Provisional Application Ser. No. 61/446,805, filed Feb. 25, 2011, the entire contents of which are hereby incorporated by reference.

### BACKGROUND OF THE INVENTION

1. Field of the Invention

The present invention relates generally to the field of human cell biology and pathology, and more particularly to diabetes. Specifically, the invention provides methods for treating diabetes and/or re-establishing normoglycemia in a subject by transplanting brown adipose tissue into the subject.

2. Description of Related Art

Type 1 diabetes (T1D), or diabetes mellitus type 1, is a form of diabetes mellitus. Type 1 diabetes is an autoimmune disease that results in the permanent destruction of insulin-producing β cells of the pancreas. Type 1 is lethal unless treatment with exogenous insulin via injections replaces the missing hormone, or a functional replacement for the destroyed pancreatic beta cells is provided (such as via a pancreas transplant).

Type 1 diabetes (formerly known as “childhood,” “juvenile” or “insulin-dependent” diabetes) is not exclusively a childhood problem. The adult incidence of type 1 is noteworthy—many adults who contract type 1 diabetes are misdiagnosed with type 2 due to the misconception of type 1 as a disease of children—and since there is no cure, all children with type 1 diabetes will grow up to be adults with type 1 diabetes.

There is currently no preventive or curative measure that can be taken against type 1 diabetes. Most people affected by type 1 diabetes are otherwise healthy and of a healthy weight when onset occurs, but they can lose weight quickly and dangerously, if not diagnosed in a relatively short amount of time. Diet and exercise cannot reverse or prevent type 1 diabetes. Although there are clinical trials ongoing that aim to find methods of preventing or slowing its development, so far none have proven successful, at least on a permanent basis.

### SUMMARY OF THE INVENTION

Thus, in accordance with the present invention, there is provided a method of treating a subject having diabetes requiring insulin treatment comprising providing to said subject brown adipose tissue. The diabetes may be other than lipoatrophic diabetes. The subject may be a human or a non-human animal. The diabetes may be type 1 diabetes, type 2 diabetes, neonatal diabetes, and MODY diabetes. In particular, the subject may suffer from autoimmune type 1 diabetes, toxin-induced type 1 diabetes, chemically-induced type 1 diabetes, or genetically-induced type 1 diabetes.

Providing may comprise surgically transplating a brown adipose tissue sample into said subject. One or more of the following diabetic symptoms may be improved by the treatment: excess gluconeogenesis, excess glycogenolysis, hyperglycemia, hyperglucagonemia, ketosis, diabetic ketoacidosis, hypertriglyceridemia, elevated plasma free fatty acid, weight loss, catabolic syndrome, terminal illness, hypertension, diabetic nephropathy, renal insufficiency, renal failure, hyperphagia, muscle wasting, diabetic neuropathy, diabetic retinopathy, or diabetic coma.

The method may further comprise administering to said subject one or more of insulin, amylin, leptin, a sulfonylurea or a GLP-1 analog. The insulin daily dosage may be 10-15%, inclusive, 5-10%, inclusive, inclusive, less than 5%, or between 0% and 5%, inclusive, of the normal daily dosage. Alternatively, the subject may receive no exogenous insulin. The subject may also be essentially devoid of endogenous insulin.

The method may achieve a venous or capillary fasting blood glucose (FBG) levels of less than 200 mg/dl, less than 175 mg/dl, less than 150 mg/dl, less than 140 mg/dl, less than 130 mg/dl, less than 126 mg/dl, less than 120 mg/dl, or less than 115 mg/dl, less than 110 mg/dl, or less than 100 mg/dl.

The BAT may be produced from donor tissue, or produced from tissue culture, such as using a cell line, an embryonic stem cell, an induced pluripotent stem cell, a hematopoietic stem cell or an adipose stem cell.

In another embodiment, there is provided a method of restoring normoglycemia in a subject diagnosed with diabetes requiring insulin treatment comprising providing to said subject brown adipose tissue. The method may further comprise administering to said subject one or more of insulin, amylin, leptin, a sulfonylurea or a GLP-1 analog. The subject may receive no more than about 10% of a normal daily dosage of insulin supplementation, or no insulin supplementation. The method may result in a venous or capillary fasting blood glucose (FBG) levels of less than 200 mg/dl, less than 175 mg/dl, less than 150 mg/dl, less than 140 mg/dl, less than 130 mg/dl, less than 126 mg/dl, less than 120 mg/dl, or less than 115 mg/dl, less than 110 mg/dl, or less than 100 mg/dl.

In yet another embodiment, there is provided a method of reducing, suppressing, attenuating, or inhibiting hyperglucogonemia or a condition associated with hyperglucogonemia in a subject having diabetes requiring insulin treatment comprising providing to said subject brown adipose tissue. The method may further comprise administering to said subject one or more of insulin, amylin, leptin, a sulfonylurea or a GLP-1 analog. The subject may receive no more than about 10% of a normal daily dosage of insulin supplementation, or no insulin supplementation. The method may result in a venous or capillary fasting blood glucose (FBG) levels of less than 200 mg/dl, less than 175 mg/dl, less than 150 mg/dl, less than 140 mg/dl, less than 130 mg/dl, less than 126 mg/dl, less than 120 mg/dl, or less than 115 mg/dl, less than 110 mg/dl, or less than 100 mg/dl.

In a further embodiment, there is provided a method of increasing adiponectin, visfatin, and/or leptin in a subject having type I diabetes comprising providing to said subject brown adipose tissue. The method may further comprise administering to said subject one or more of insulin, amylin, leptin, a sulfonylurea or a GLP-1 analog. The subject may receive no more than about 10% of a normal daily dosage of insulin supplementation, or no insulin supplementation. The method may result in a venous or capillary fasting blood glucose (FBG) levels of less than 200 mg/dl, less than 175 mg/dl, less than 150 mg/dl, less than 140 mg/dl, less than 130 mg/dl, less than 126 mg/dl, less than 120 mg/dl, or less than 115 mg/dl, less than 110 mg/dl, or less than 100 mg/dl.

An additional embodiment comprises a method of treating pre-diabetic metabolic syndrome in a subject comprising providing to said subject brown adipose tissue.

As used herein the specification, “a” or “an” may mean one or more. As used herein in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C. Subcutaneous transplantation of embryonic brown adipose tissue results in improved glucose homeostasis and weight gain. Basal non-fasting blood glucose (FIG. 1A) and body weight (FIG. 1B). Horizontal dashed line in FIG. 1A represents baseline glucose levels in non-diabetic mice of 146.13±7.36 mg/dl. FIG. 1A: p<0.05, between BAT transplant and untreated diabetic controls at each time point. FIG. 1B: p<0.05 at 8 weeks post-transplant. FIG. 1C: Glucose tolerance tests on BAT transplant, normal, and diabetic control mice. * p<0.05 when compared with untreated diabetic controls or diabetic pre-transplant condition. Normal, pre-transplant and 1 month post-transplant (n=9); 6 months post-transplant (n=3); untreated diabetic controls (n=5).

FIGS. 2A-F. Glucose control is independent of insulin. FIG. 2A: Plasma insulin response to glucose challenge at 3 months post-transplants (n=5), diabetic pre-transplants (n=9), untreated diabetic (n=5) and normal non-diabetic controls (n=7). p<0.05, at 15 and 30 min between normal control and all other groups. FIGS. 2B-D: Insulin immunofluorescence of pancreatic sections from 3 months post BAT transplant mice (FIG. 2D) compared with those of normal (FIG. 2B) and untreated diabetic (FIG. 2C) controls. Scale bar=50 μm. FIG. 2E: First Phase insulin release during IPGTT 1 through 3 months post-transplant, compared with normal and diabetic controls (n=3). *p<0.05, when compared with all other groups at 1 and 3 minute points. FIG. 2F. Arginine stimulated insulin release 1 and 3 months post-transplant, compared with normal and diabetic controls (n=4). * p<0.05, when compared with all other groups at each time point.

FIGS. 3A-D. BAT Transplant failure is associated with inflammatory changes in adipose tissue. Histological sections of replenished white adipose tissue in euglycemic mouse 3 months posttransplant (FIG. 3C) is compared with normal (FIG. 3A) and diabetic (FIG. 3B) controls, and a transplant recipient that reverted to diabetes at 4 months (FIG. 3D). Sections are immunostained for TNFα (green) and IL6 (red). All tissues contain some green autofluorescence, and TNFα expression is indicated by bright green spots: TNFα immunofluorescence appears near the right border in the normal section (FIG. 3A), towards the bottom in the TP (FIG. 3C); throughout the section in FIG. 3B & FIG. 3D, co-localizing with IL6 and appearing yellow-orange. In addition to the presence of larger amounts of inflammatory markers, the failed adipose tissue shows other signs of inflammation such as enlargement of adipocytes and infiltration of macrophages resulting in thickening of cell membranes. Sections shown are representative of 4 BAT transplants, 2 untreated diabetic controls and 2 normal animals analyzed. Scale bar=50 μM.

FIGS. 4A-F. Long-term glucose control shows a correlation with adipokines rather than insulin. FIGS. 4A-D: Fasting blood glucose (FIG. 4A), glucose-stimulated insulin (FIG. 4B), adiponectin (FIG. 4C), and leptin (FIG. 4D) levels over time from BAT transplant mice are shown. While insulin levels in BAT transplant mice remain consistently subnormal, adiponectin and leptin levels show fluctuations that mirror the changes in blood glucose. FIGS. 4A-C: normal (n=5), BAT transplant and diabetic (n=7); FIG. 4D: normal (n=5), BAT transplant (n=7), diabetic (n=6); FIG. 4A: p<0.0005 when diabetic and pre-transplant (0 weeks) groups are compared with all other groups. FIG. 4B: p<0.005 between normal control and all other groups. FIG. 4C: p<0.001 between BAT transplants and pre-transplant (0 weeks) except for 16 weeks; p<0.001 between BAT transplants and untreated diabetic controls at 8, 12 and 24 weeks. FIG. 4D: p<0.05 between BAT transplants and normal, diabetic and pretransplant controls at 4, 8, 20 weeks, and p<0.005 at 24 weeks post-transplant. FIGS. 4E-F: Plasma glucagon (FIG. 4E) and IGF-1 (FIG. 4F) levels from 5 & 6 month BAT transplants compared with normal and diabetic controls. BAT transplants are associated with a decrease of glucagon and increase of IGF-1 (n=4). FIG. 4E: p<0.0005 between BAT transplants and diabetic controls; p<0.05 between BAT transplants and normal controls. FIG. 4F: p<0.05 between BAT transplants and diabetic or normal controls.

FIGS. 5A-B. Inhibition of insulin receptor impairs glucose tolerance in euglycemic transplant mice. S981, a competitive antagonist of the insulin receptor, was administered immediately prior to GTT. S961 resulted in impairment of glucose tolerance in both normal controls (FIG. 5A: n=4) and BAT transplant recipients euglycemic at 3 months (FIG. 5B: n=5). FIG. 5A: p<0.05 when 60 and 120 min time points are compared with and without S961. FIG. 5B: p<0.05 when 120 min time point is compared with and without S961.

FIGS. 6A-C. Subcutaneous transplantation of embryonic brown adipose tissue results in improvement of glucose and insulin tolerance in C57BL/6J mice. FIG. 6A: Glucose tolerance tests before and at monthly intervals after transplants, compared with normal and diabetic controls. *=p<0.05 when compared with untreated diabetic controls or diabetic pre-transplant condition (Normal, Pre-transplant, and transplants up to 3 months: n=9; 5 months: n=5; 6 months: n=3; Untreated diabetic: n=5). FIG. 6B: Progressive decline of glucose tolerance in untreated diabetic control mice. n=4; *=p<0.05, when 3 month data are compared with 1 or 2 months. FIG. 6C: Insulin tolerance in successful BAT transplant mice euglycemic at 2 or 3 months, compared with normal and diabetic controls in C57BL/6J mice. *=p<0.05, compared with both other groups. n=4 for BAT transplants; n=3 for normal and diabetic groups.

FIGS. 7A-D. Subcutaneous transplantation of embryonic brown adipose tissue results in improvement of glucose homeostasis and body weight in NCRNU nude mice. Basal non-fasting blood glucose (FIG. 7A) and body weight (FIG. 7B) over time. The horizontal dashed line indicates the measured baseline glucose levels in non-diabetic Nude mice 101.5±5.01 mg/dl. p<0.05, when compared with untreated diabetic controls at 3 months post-transplant. FIG. 7C: Glucose tolerance tests before and at monthly intervals after transplants, compared with normal and diabetic controls. For FIG. 7A-C, normal mouse group: n=10; BAT Transplants and Untreated diabetic groups: n=6. FIG. 7D: Insulin tolerance in successful BAT transplant mice euglycemic at 2 or 3 months, compared with normal and diabetic controls in nude mice. For FIG. 7D, n=4 for BAT transplants; n=3 for normal and diabetic groups. *=p<0.05, and **=p<0.005 when compared with untreated diabetic controls or diabetic pre-transplant condition.

FIG. 8A-B. Failure to achieve euglycemia is associated with adipose tissue inflammation. Adipose tissue sections from C57BL/6J mice with failed BAT transplants that did not achieve euglycemia in 4 months. Sections are immunostained for TNFα (green) and IL6 (red). Scale bar=50 μm. All tissues contain some green autofluorescence, and TNFα expression is indicated by bright green spots, sometimes co-localizing with IL6 and appearing yellow-orange. In addition to the presence of large amounts of inflammatory markers, the failed adipose tissue shows other signs of inflammation such as enlargement of adipocytes and thickening of cell membranes. Sections shown are representative 3 failed BAT transplants analyzed.

FIG. 9. Plasma adiponectin levels during glucose tolerance test in C57BL/6J mice. Adiponectin levels are compared in blood samples taken during IPGTT in normal and diabetic control groups, and mice that are euglycemic following BAT transplants. No significant acute response to glucose is observed.

FIGS. 10A-B. Inhibition of insulin receptor impairs glucose tolerance in euglycemic BAT transplant NCRNU nude mice. S981, a competitive inhibitor of insulin receptor, was administered immediately prior to GTT. S961 resulted in impairment of glucose tolerance in both normal controls (FIG. 10A: n=3) and BAT transplant recipients euglycemic at 3 months (FIG. 10B: n=4). FIG. 10A: p<0.05 when 30, 60 and 120 min. time points are compared with and without S961. FIG. 10B: p<0.05 when 60 and 120 min. time points are compared with and without S961.

FIGS. 11A-D. Progress of BAT transplant monitored by UCP-1 immunostaining in transplant site at different time points. UCP-1 appears brown with DAB staining in H&E sections. FIG. 11A: Control, fresh embryonic BAT prior to transplant; FIGS. 11B-D: 2,3 and 6 months after transplant. Scale bar=100 μm.

FIGS. 12A-C. IGF-1 immuno-staining at the BAT transplant site 3 months post transplant. IGF-1 appears brown with DAB staining in H&E sections. FIG. 12A: Transplanted BAT; FIG. 12B: Surrounding WAT; FIG. 12C: Area showing BAT developing into WAT. Scale bar=100 μm.

### DESCRIPTION OF ILLUSTRATIVE EMBODIMENTS

As discussed herein, the inventors have now demonstrated that transplantation of brown adipose tissue (BAT) reversed diabetes in a fashion similar to observed following pancreatic bud transplant. None of the other transplanted tissue types gave positive results. This phenomenon is observed in ˜70% of mouse recipients from of two different strains (either C57BL/6 or NCRNU-M-M nude mice; n>25). There is no change in their pancreatic insulin content or plasma insulin, and the recovery is accompanied by reduced inflammatory cytokines in the animals' native adipose tissue. These data suggest a new paradigm where the BAT transplant affects whole animal physiology in a way that lowers inflammation in the native white adipose tissue (and perhaps other tissues). Such a role for brown adipose tissue is currently an active research area (e.g., Tran & Kahn, 2010). In theory, BAT results in removal of inflammation that then allows the white adipose tissue to secrete adipokines that act together to restore normal blood glucose levels. This action appears to be fundamentally different from that of insulin, which increases acutely in the bloodstream in response to caloric intake. In contrast, expression of the adipokines appears to be upregulated continuously. Recent literature supports a key role for leptin in restoration of normal glycemia, but the inventors have not observed the 2-fold increase in IGF seen in leptin treatment (Yu et al., 2008). Thus, the inventors' working model is that the action of adipokines (likely combined with other factors) establishes a new equilibrium in the animal that allows for chronic glycemic control without the addition of exogenous insulin. These and other aspects of the invention are described in greater detail below.

### I. DIABETES

A. Type 1 Diabetes

Type 1 diabetes (T1D), or diabetes mellitus type 1, is a form of diabetes mellitus. Type 1 diabetes is an autoimmune disease that results in the permanent destruction of insulin-producing β cells of the pancreas. Type 1 is lethal unless treatment with exogenous insulin via injections replaces the missing hormone, or a functional replacement for the destroyed pancreatic beta cells is provided (such as via a pancreas transplant).

Type 1 diabetes (formerly known as “childhood,” “juvenile” or “insulin-dependent” diabetes) is not exclusively a childhood problem. The adult incidence of type 1 is noteworthy—many adults who contract type 1 diabetes are misdiagnosed with type 2 due to the misconception of type 1 as a disease of children—and since there is no cure, all children with type 1 diabetes will grow up to be adults with type 1 diabetes.

There is currently no preventive measure that can be taken against type 1 diabetes. Most people affected by type 1 diabetes are otherwise healthy and of a healthy weight when onset occurs, but they can lose weight quickly and dangerously, if not diagnosed in a relatively short amount of time. Diet and exercise cannot reverse or prevent type 1 diabetes. Although there are clinical trials ongoing that aim to find methods of preventing or slowing its development, so far none have proven successful, at least on a permanent basis.

The most useful laboratory test to distinguish type 1 from type 2 diabetes is the C-peptide assay, which is a measure of endogenous insulin production since external insulin (to date) has included no C-peptide. However, C-peptide is not absent in type 1 diabetes until insulin production has fully ceased, which may take months. The presence of anti-islet antibodies (to Glutamic Acid Decarboxylase, Insulinoma Associated Peptide-2 or insulin), or lack of insulin resistance, determined by a glucose tolerance test, would also be suggestive of type 1. As opposed to that, many type 2 diabetics still produce insulin internally, and all have some degree of insulin resistance. Testing for GAD 65 antibodies has been proposed as an improved test for differentiating between type 1 and type 2 diabetes.

The cause of type 1 diabetes is still not fully understood. Some theorize that type 1 diabetes could be a virally-induced autoimmune response. Autoimmunity is a condition where one's own immune system “attacks” structures in one's own body either destroying the tissue or decreasing its functionality. In the proposed scenario, pancreatic beta cells in the Islets of Langerhans are destroyed or damaged sufficiently to abolish endogenous insulin production. This etiology makes type 1 distinct from type 2 diabetes mellitus. It should also be noted that the use of insulin in a patient's diabetes treatment protocol does not render them as having type 1 diabetes, the type of diabetes a patient has is determined only by disease etiology. The autoimmune attack may be triggered by reaction to an infection, for example by one of the viruses of the Coxsackie virus family or German measles, although the evidence is inconclusive.

This vulnerability is not shared by everyone, for not everyone infected by these organisms develops type 1 diabetes. This has suggested a genetic vulnerability and there is indeed an observed inherited tendency to develop type 1. It has been traced to particular HLA genotypes, though the connection between them and the triggering of an auto-immune reaction is poorly understood. Wide-scale genetic studies have shown links between genetic vulnerabilities for type 1 diabetes and Multiple Sclerosis and Crohn's Disease.

Some researchers believe that the autoimmune response is influenced by antibodies against cow's milk proteins. A large retrospective controlled study published in 2006 strongly suggests that infants who were never breastfed had a risk for developing type 1 diabetes twice that of infants who were breastfed for at least three months. The mechanism, if any, is not understood. No connection has been established between autoantibodies, antibodies to cow's milk proteins, and type 1 diabetes. A subtype of type 1 (identifiable by the presence of antibodies against β cells) typically develops slowly and so is often confused with type 2 diabetes (see below). In addition, a small proportion of type 1 cases have the hereditary condition maturity onset diabetes of the young (MODY; see below) which can also be confused with type 2.

Vitamin D in doses of 2000 IU per day given during the first year of a child's life has been connected in one study in Northern Finland (where intrinsic production of Vitamin D is low due to low natural light levels) with an 80% reduction in the risk of getting type 1 diabetes later in life. Some suggest that deficiency of Vitamin D₃ (one of several related chemicals with Vitamin D activity) may be an important pathogenic factor in type 1 diabetes independent of geographical latitude.

Some chemicals and drugs specifically destroy pancreatic cells. Vacor (N-3-pyridylmethyl-N′-p-nitrophenyl urea), a rodenticide introduced in the United States in 1976, selectively destroys pancreatic β cells, resulting in type 1 diabetes after accidental or intentional ingestion. Vacor was withdrawn from the U.S. market in 1979. Zanosar® is the trade name for streptozotocin, an antibiotic and antineoplastic agent used in chemotherapy for pancreatic cancer, that kills β cells, resulting in loss of insulin production.

Other pancreatic problems, including trauma, pancreatitis or tumors (either malignant or benign), can also lead to loss of insulin production. The exact cause(s) of type 1 diabetes are not yet fully understood, and research on those mentioned, and others, continues.

B. T1D Treatments

Untreated type 1 diabetes can lead to one form of diabetic coma, diabetic ketoacidosis, which can be fatal. Other aspects of the disease include excess gluconeogenesis, excess glycogenolysis, hyperglycemia, hyperglucagonemia, ketosis, hypertriglyceridemia, elevated plasma free fatty acid, weight loss, hypertension, diabetic nephropathy, renal insufficiency, renal failure, hyperphagia, muscle wasting, diabetic neuropathy, and diabetic retinopathy

Type 1 is treated with insulin replacement therapy—usually by injection or insulin pump, along with attention to dietary management, typically including carbohydrate tracking, and careful monitoring of blood glucose levels using glucose meters. At present, insulin treatment must be continued for a lifetime; this will change if better treatment, or a cure, is discovered. Continuous glucose monitors have been developed which can alert patients to the presence of dangerously high or low blood sugar levels, but the lack of widespread insurance coverage has limited the impact these devices have had on clinical practice so far.

There are both short- and long-term disadvantages to insulin therapy. The main short-term issue concern with insulin monotherapy is the instability of the daily glucose profiles achieved by peripheral injections of insulin. Even optimally controlled patients with at target HgbA1c values have daily spikes of hyperglycemia, with occasional hypoglycemic dips. This may be the result of the enormous anatomical disadvantage of peripherally injected insulin, which cannot meet the high insulin requirements of proximal targets such as alpha cells and hepatocytes without far exceeding the insulin requirements of distal targets such as muscle and fat. The intra-islet concentration of endogenous insulin that perfuses alpha cells in normal islets has been estimated to be over 20× higher than the levels generated by peripheral injection, and the concentration of endogenous insulin perfusing the liver is 4- to 5-times higher. This means that even a high concentration of exogenous insulin in peripheral plasma may not approach the physiologic levels of endogenous insulin that perfuse these two proximal insulin targets, which control endogenous glucose production.

A second important disadvantage of injected insulin is its inability to respond on a minute-to-minute basis to changes in need, in particular, to lower it instantly when glucose levels are falling. These facts suggest that, if the wild and inappropriate oscillations of insulin and glucagon that create inappropriate swings in hepatic glucose production could be chronically stabilized by suppressing glucagon independently of insulin, the total daily dose of insulin could be reduced to levels that would manage postprandial hyperglycemia and nothing else. The hope is that this would establish a more stable pattern of glucoregulation.

A third important contributing factor to glucose lability is lipolytic lability, which intermittently floods the target tissues of insulin with fatty acids that impair sensitivity to insulin action on glucose metabolism. This contributes to instability of glucose levels, which can fluctuate from dangerously low levels of hypoglycemia to undesirably high hyperglycemia, making frequent blood glucose determination and multiple insulin injections mandatory, thereby significantly lowering the quality of life for patients.

The major long-term effect of insulin therapy is insulin resistance, a well characterized component of type 1 diabetes. As in T2DM, the degree of insulin resistance is closely associated with risk of cardiovascular disease. The high prevalence of coronary artery disease among patients with T1DM is traditionally ascribed to the disease rather than to life-long insulin monotherapy. The role of insulin in the macrovascular complications of T1DM deserves to be examined more closely, given the relationship between the diet-driven endogenous hyperinsulinemia of obesity and the metabolic syndrome, particularly in insulin-resistant patients treated with U-500 insulin. Insulin is a powerful lipogenic force; a life-time of exogenous hyperinsulinemia in T1DM could also cause a form of metabolic syndrome, with insulin resistance, hyperlipidemia, hypercholesterolemia, coronary artery disease and lipotoxic cardiomyopathy and occasionally obesity.

In more extreme cases, a pancreas transplant can help restore proper glucose regulation. However, the surgery and accompanying immunosuppression required is considered by many physicians to be more dangerous than continued insulin replacement therapy and is therefore often used only as a last resort (such as when a kidney must also be transplanted or in cases where the patient's blood glucose levels are extremely volatile). Experimental replacement of β cells (by transplant or from stem cells) is being investigated in several research programs and may become clinically available in the future. Thus far, β cell replacement has only been performed on patients over age 18, and with tantalizing successes amidst nearly universal failure.

Pancreas transplants are generally recommended if a kidney transplant is also necessary. The reason for this is that introducing a new kidney requires taking immunosuppressive drugs anyway, and this allows the introduction of a new, functioning pancreas to a patient with diabetes without any additional immunosuppressive therapy. However, pancreas transplants alone can be wise in patients with extremely labile type 1 diabetes mellitus.

Less invasive than a pancreas transplant, islet cell transplantation is currently the most highly used approach in humans to temporarily cure type 1 diabetes. In one variant of this procedure, islet cells are injected into the patient's liver, where they take up residence and begin to produce insulin. The liver is expected to be the most reasonable choice because it is more accessible than the pancreas, and the islet cells seem to produce insulin well in that environment. The patient's body, however, will treat the new cells just as it would any other introduction of foreign tissue. The immune system will attack the cells as it would a bacterial infection or a skin graft. Thus, the patient also needs to undergo treatment involving immunosuppressants, which reduce immune system activity.

Recent studies have shown that islet cell transplants have progressed to the point that 58% of the patients in one study were insulin independent one year after the operation. Ideally, it would be best to use islet cells which will not provoke this immune reaction, but investigators are also looking into placing islets into a protective coating which enables insulin to flow out while protecting the islets from white blood cells.

C. Type 2 Diabetes

Diabetes mellitus type 2—formerly non-insulin-dependent diabetes mellitus (NIDDM) or adult-onset diabetes—is a metabolic disorder that is characterized by high blood glucose in the context of insulin resistance and relative insulin deficiency. Diabetes is often initially managed by increasing exercise and dietary modification. As the condition progresses, medications may be needed. Unlike type 1 diabetes, there is very little tendency toward ketoacidosis though it is not unheard of. One effect that can occur is non-ketonic hyperglycemia. Long-term complications from high blood sugar can include increased risk of heart attacks, strokes, amputation, and kidney failure. The classical symptoms of diabetes are polyuria (frequent urination), polydipsia (increased thirst), polyphagia (increased hunger), fatigue and weight loss.

Type 2 diabetes is due primarily to lifestyle factors and genetics. It was also found that oligomers of islet amyloid polypeptide (IAPP), a protein that forms amyloid deposits in the pancreas during type 2 diabetes, triggered the NLRP3 inflammasome and generated mature IL-1β. One therapy for type 2 diabetes, glyburide, suppressed IAPP-mediated IL-1β production in vitro.

A number of lifestyle factors are known to be important to the development of type 2 diabetes. In one study, those who had high levels of physical activity, a healthy diet, did not smoke, and consumed alcohol in moderation had an 82% lower rate of diabetes. When a normal weight was included the rate was 89% lower. In this study a healthy diet was defined as one high in fiber, with a high polyunsaturated to saturated fat ratio, and a lower mean glycemic index. Obesity has been found to contribute to approximately 55% of cases of type 2 diabetes, and decreasing consumption of saturated fats and trans fatty acids while replacing them with unsaturated fats may decrease the risk. The increased rate of childhood obesity in between the 1960s and 2000s is believed to have led to the increase in type 2 diabetes in children and adolescents.

There are many factors which can potentially give rise to or exacerbate type 2 diabetes. These include obesity, hypertension, elevated cholesterol (combined hyperlipidemia), and with the condition often termed metabolic syndrome (it is also known as Syndrome X, Reavan's syndrome, or CHAOS). Other causes include acromegaly, Cushing's syndrome, thyrotoxicosis, pheochromocytoma, chronic pancreatitis, cancer and drugs. Additional factors found to increase the risk of type 2 diabetes include aging, high-fat diets and a less active lifestyle. There is also a strong inheritable genetic connection in type 2 diabetes: having relatives (especially first degree) with type 2 increases risks of developing type 2 diabetes very substantially. In addition, there is also a mutation to the Islet Amyloid Polypeptide gene that results in an earlier onset, more severe, form of diabetes.

About 55 percent of type 2 diabetes patients are obese at diagnosis—chronic obesity leads to increased insulin resistance that can develop into type 2 diabetes, most likely because adipose tissue (especially that in the abdomen around internal organs) is a (recently identified) source of several chemical signals to other tissues (hormones and cytokines). Other research shows that type 2 diabetes causes obesity as an effect of the changes in metabolism and other deranged cell behavior attendant on insulin resistance.

However, environmental factors (almost certainly diet and weight) play a large part in the development of type 2 diabetes in addition to any genetic component. This can be seen from the adoption of the type 2 diabetes epidemiological pattern in those who have moved to a different environment as compared to the same genetic pool who have not. Immigrants to Western developed countries, for instance, as compared to lower incidence countries of origins.

There is a stronger inheritance pattern for type 2 diabetes. Those with first-degree relatives with type 2 diabetes have a much higher risk of developing type 2 diabetes, increasing with the number of those relatives. Concordance among monozygotic twins is close to 100%, and about 25% of those with the disease have a family history of diabetes. Genes significantly associated with developing type 2 diabetes, include TCF7L2, PPARG, FTO, KCNJ11, NOTCH2, WFS1, CDKAL1, IGF2BP2, SLC30A8, JAZF1, and HHEX. KCNJ11 (potassium inwardly rectifying channel, subfamily J, member 11), encodes the islet ATP-sensitive potassium channel Kir6.2, and TCF7L2 (transcription factor 7—like 2) regulates proglucagon gene expression and thus the production of glucagon-like peptide-1. Moreover, obesity (which is an independent risk factor for type 2 diabetes) is strongly inherited. Gene expression promoted by a diet of fat and glucose as well as high levels of inflammation related cytokines found in the obese results in cells that produce fewer and smaller mitochondria than is normal, and are thus prone to insulin resistance.

Some drugs, used for any of several conditions, can interfere with the insulin regulation system, possibly producing drug induced hyperglycemia. Some examples follow, giving the biochemical mechanism in each case: atypical antipsychotics; beta-blockers; calcium channel blockers; corticosteroids; fluoroquinolones; niacin—causes increased insulin resistance due to increased free fatty acid mobilization; phenothiazines; protease inhibitors; somatropin; and thiazide diuretics.

Insulin resistance means that body cells do not respond appropriately when insulin is present. Unlike type 1 diabetes mellitus, insulin resistance is generally “post-receptor,” meaning it is a problem with the cells that respond to insulin rather than a problem with the production of insulin.

This is a more complex problem than type 1, but is sometimes easier to treat, especially in the early years when insulin is often still being produced internally. Severe complications can result from improperly managed type 2 diabetes, including renal failure, erectile dysfunction, blindness, slow healing wounds (including surgical incisions), and arterial disease, including coronary artery disease. The onset of type 2 diabetes has been most common in middle age and later life, although it is being more frequently seen in adolescents and young adults due to an increase in child obesity and inactivity. A type of diabetes called MODY (see below) is increasingly seen in adolescents, but this is classified as a diabetes due to a specific cause and not as type 2 diabetes.

The World Health Organization definition of diabetes is for a single raised glucose reading with symptoms, otherwise raised values on two occasions, of either:
fasting plasma glucose ≧7.0 mmol/l (126 mg/dl); or
with a glucose tolerance test, two hours after the oral dose a plasma glucose ≧11.1 mmol/l (200 mg/dl)

Type 2 diabetes risk can be reduced in many cases by making changes in diet and increasing physical activity. The American Diabetes Association (ADA) recommends maintaining a healthy weight, getting at least 2½ hours of exercise per week (several brisk sustained walks appear sufficient), having a modest fat intake, and eating sufficient fiber (e.g., from whole grains). There is inadequate evidence that eating foods of low glycemic index is clinically helpful despite recommendations and suggested diets emphasizing this approach.

Some studies have shown delayed progression to diabetes in predisposed patients through prophylactic use of metformin, rosiglitazone, or valsartan. In patients on hydroxychloroquine for rheumatoid arthritis, incidence of diabetes was reduced by 77% though causal mechanisms are unclear. Lifestyle interventions are however more effective than metformin at preventing diabetes regardless of weightloss.

Left untreated, type 2 diabetes is a chronic, progressive condition, but there are well-established treatments which can delay or prevent entirely the formerly inevitable consequences of the condition. Often, the condition is viewed as progressive since poor management of blood sugar leads to a myriad of steadily worsening complications. However, if blood sugar is properly maintained, then the condition is, in a limited sense, cured—that is, patients are at no heightened risk for neuropathy, blindness, or any other high blood sugar complication, though the underlying issue, a tendency to hyperglycemia has not been addressed directly.

There are two main goals of treatment: reduction of mortality and concomitant morbidity (from assorted diabetic complications) and preservation of quality of life. The first goal can be achieved through close glycemic control (i.e., to near normal blood glucose levels); the reduction in severity of diabetic side effects has been very well demonstrated in several large clinical trials and is established beyond controversy. The second goal is often addressed (in developed countries) by support and care from teams of diabetic health workers (usually physician, PA, nurse, dietitian or a certified diabetic educator). Endocrinologists, family practitioners, and general internists are the physician specialties most likely to treat people with diabetes. Knowledgeable patient participation is vital to clinical success, and so patient education is a crucial aspect of this effort.

Type 2 diabetes is initially treated by adjustments in diet and exercise, and by weight loss, most especially in obese patients. The amount of weight loss which improves the clinical picture is sometimes modest (2-5 kg or 4.4-11 lb); this is almost certainly due to currently poorly understood aspects of fat tissue activity, for instance chemical signaling (especially in visceral fat tissue in and around abdominal organs). In many cases, such initial efforts can substantially restore insulin sensitivity. In some cases strict diet can adequately control the glycemic levels.

There are several drugs available for type 2 diabetics—most are unsuitable or even dangerous for use by type 1 diabetics. They fall into several classes and are not equivalent, nor can they be simply substituted one for another. All are prescription drugs.

One of the most widely used drugs now used for type 2 diabetes is the biguanide metformin; it works primarily by reducing liver release of blood glucose from glycogen stores and secondarily by provoking some increase in cellular uptake of glucose in body tissues. Metformin also reduces insulin resistance and is preferred in obese patients as it promotes weight loss. Both historically and currently the most commonly used drugs are in the Sulfonylurea group, of which several members (including glibenclamide and gliclazide) are widely used; these increase glucose stimulated insulin secretion by the pancreas and so lower blood glucose even in the face of insulin resistance. Their chief adverse effect is increased chance of hypoglycemic episodes.

Newer drug classes include:
thiazolidinediones (TZDs) (rosiglitazone, pioglitazone, and troglitazone—the last, as Rezulin, was withdrawn from the US market because of an increased risk of systemic acidosis). These increase tissue insulin sensitivity by affecting gene expression
α-glucosidase inhibitors (acarbose and miglitol) which interfere with absorption of some glucose containing nutrients, reducing (or at least slowing) the amount of glucose absorbed
meglitinides which stimulate insulin release (nateglinide, repaglinide, and their analogs) quickly; they can be taken with food, unlike the sulfonylureas which must be taken prior to food (sometimes some hours before, depending on the drug)
peptide analogs which work in a variety of ways:
incretin mimetics which increase insulin output from the beta cells among other effects. These includes the Glucagon-like peptide (GLP) analog exenatide, sometimes referred to as lizard spit as it was first identified in Gila monster saliva
dipeptidyl peptidase-4 (DPP-4) inhibitors increase Incretin levels (sitagliptin) by decreasing their deactivation rates
amylin agonist analog, which slows gastric emptying and suppresses glucagon (pramlintide)
   In rare cases, if antidiabetic drugs fail (i.e., the clinical benefit stops), insulin therapy may be necessary—usually in addition to oral medication therapy—to maintain normal or near normal glucose levels. Typical total daily dosage of insulin is 0.6 U/kg. But, of course, best timing and indeed total amounts depend on diet (composition, amount, and timing) as well the degree of insulin resistance. More complicated estimations to guide initial dosage of insulin are:
for men, [(fasting plasma glucose [mmol/liter]−5)×2]×(weight [kg]÷(14.3×height [m])−height [m])
for women, [(fasting plasma glucose [mmol/liter]−5)×2]×(weight [kg]±(13.2×height [m])−height [m])
   The initial insulin regimen are often chosen based on the patient's blood glucose profile.

Initially, adding nightly insulin to patients failing oral medications may be best. Nightly insulin combines better with metformin than with sulfonylureas. The initial dose of nightly insulin (measured in IU/d) should be equal to the fasting blood glucose level (measured in mmol/L). If the fasting glucose is reported in mg/dl, multiply by 0.05551 to convert to mmol/L. When nightly insulin is insufficient, choices include: premixed insulin with a fixed ratio of short and intermediate acting insulin; this tends to be more effective than long acting insulin, but is associated with increased hypoglycemia. Initial total daily dosage of biphasic insulin can be 10 units if the fasting plasma glucose values are less than 180 mg/dl or 12 units when the fasting plasma glucose is above 180 mg/dl. A guide to titrating fixed ratio insulin is available. Insulin Pump therapy in type 2 diabetes is gradually becoming popular. In an original published study, in addition to reduction of blood sugars, there is evidence of profound benefits in resistant neuropathic pain and also improvements in sexual performance.

D. MODY or NIDDM

Maturity onset diabetes of the young (MODY)] refers to any of several hereditary forms of diabetes caused by mutations in an autosomal dominant gene (sex independent, i.e., inherited from any of the parents) disrupting insulin production. MODY is often referred to as “monogenic diabetes” to distinguish it from the more common types of diabetes (especially type 1 and type 2), which involve more complex combinations of causes involving multiple genes (i.e., “polygenic”) and environmental factors. MODY 2 and MODY 3 are the most common forms. The severity of the different types varies considerably, but most commonly MODY acts like a very mild version of type 1 diabetes, with continued partial insulin production and normal insulin sensitivity. MODY is not type 2 diabetes in a young person, as might erroneously be inferred from the name.

The term MODY dates back to 1964, when diabetes mellitus was considered to have two main forms: juvenile-onset and maturity-onset, which roughly corresponded to what the inventors now call type 1 and type 2. MODY was originally applied to any child or young adult who had persistent, asymptomatic hyperglycemia without progression to diabetic ketosis or ketoacidosis. In retrospect, the inventors can now recognize that this category covered a heterogeneous collection of disorders which included cases of dominantly inherited diabetes (the topic of this article, still called MODY today), as well as cases of what the inventors would now call type 2 diabetes occurring in childhood or adolescence, and a few even rarer types of hyperglycemia (e.g., mitochondrial diabetes or mutant insulin). Many of these patients were treated with sulfonylureas with varying degrees of success.

By the 1990's, as the understanding of the pathophysiology of diabetes has improved, the concept and usage of “MODY” have become refined and narrower. It is now used as a synonym for dominantly inherited, monogenic defects of insulin secretion occurring at any age, and no longer includes any forms of type 2 diabetes.

There are two general types of clinical presentation. Some forms of MODY produce significant hyperglycemia and the typical signs and symptoms of diabetes: increased thirst and urination (polydipsia and polyuria). In contrast, however, many people with MODY have no signs or symptoms and are diagnosed by either accident, when a high glucose is discovered during testing for other reasons, or screening of relatives of a person discovered to have diabetes. Discovery of mild hyperglycemia during a routine glucose tolerance test for pregnancy is particularly characteristic.

MODY cases may make up as many as 5% of presumed type 1 and type 2 diabetes cases in a large clinic population. While the goals of diabetes management are the same no matter what type, there are two primary advantages of confirming a diagnosis of MODY. Firstly, insulin may not be necessary and it may be possible to switch a person from insulin injections to oral agents without loss of glycemic control. Secondly, it may prompt screening of relatives and so help identify other cases in family members. As it occurs infrequently, many cases of MODY are initially assumed to be more common forms of diabetes: type 1 if the patient is young and not overweight, type 2 if the patient is overweight, or gestational diabetes if the patient is pregnant. Standard diabetes treatments (insulin for type 1 and gestational diabetes, and oral hypoglycemic agents for type 2) are often initiated before the doctor suspects a more unusual form of diabetes.

In some forms of MODY, standard treatment is appropriate, though exceptions occur. In MODY2, oral agents are relatively ineffective and insulin is unnecessary. In MODY1 and MODY3, insulin may be more effective than drugs to increase insulin sensitivity. Sulfonylureas are effective in the KATP channel forms of neonatal-onset diabetes.

The following characteristics suggest the possibility of a diagnosis of MODY in hyperglycemic and diabetic patients:
Mild to moderate hyperglycemia (typically 130-250 mg/dl, or 7-14 mM) discovered before 30 years of age. However, anyone under 50 can develop MODY.
A first degree relative with a similar degree of diabetes.
Absence of positive antibodies or other autoimmunity (e.g., thyroiditis) in patient and family.
Persistence of a low insulin requirement (e.g., less than 0.5 u/kg/day) past the usual “honeymoon” period.
Absence of obesity (although overweight or obese people can get MODY), or other problems associated with type 2 diabetes or metabolic syndrome (e.g., hypertension, hyperlipidemia, polycystic ovary syndrome).
Insulin resistance very rarely happens.
Cystic kidney disease in patient or close relatives.
Non-transient neonatal diabetes, or apparent type 1 diabetes with onset before 6 months of age.
   The diagnosis of MODY is confirmed by specific gene testing, now available through several commercial laboratories.

The recognised forms of MODY are all due to ineffective insulin production or release by pancreatic β-cells. Several of the defects are mutations of transcription factor genes. One form is due to mutations of the glucokinase gene. For each form of MODY, multiple specific mutations involving different amino acid substitutions have been discovered. In some cases, there are significant differences in the activity of the mutant gene product that contribute to variations in the clinical features of the diabetes (such as degree of insulin deficiency or age of onset). Some sources make a distinction between two different forms of monogenetic diabetes: MODY and neonatal diabetes. However, they have much in common, and are often studied together.

Unfortunately, chronic hyperglycemia of any cause can eventually cause blood vessel damage and the microvascular complications of diabetes. The principal treatment goals for people with MODY—keeping the blood sugars as close to normal as possible (“good glycemic control”), while minimizing other vascular risk factors—are the same for all known forms of diabetes. Tools available for management are also those available for all forms of diabetes: blood testing, changes in diet, physical exercise, oral hypoglycemic agents, and insulin injections. In many cases these goals can be achieved more easily with MODY than with ordinary types 1 and 2 diabetes. Some people with MODY may require insulin injections to achieve the same glycemic control that another person may attain with careful eating or an oral medication. When oral hypoglycemic agents are used in MODY, the sulfonylureas remain the oral medication of first resort. Patients with MODY less often suffer from obesity and insulin resistance than those with ordinary type 2 diabetes (for whom insulin sensitizers like metformin or the thiazolidinediones are often preferred over the sulfonylureas).

E. Neonatal Diabetes

A newly identified and potentially treatable form of monogenic diabetes is the neonatal diabetes caused by activating mutations of the KCNJ11 gene, which codes for the Kir6.2 subunit of the beta cell KATP channel. It can be associated with GCK, KCNJ11, INS, and ABCC8. This results in congenital impairment of insulin release, although in the past, this was always being thought to be unusually early type 1 diabetes mellitus. The insulin deficiency results in intrauterine growth retardation with birth weight small for gestational age. The diabetes is usually diagnosed in the first 3 months of life due to continuing poor weight gain, polyuria, or diabetic ketoacidosis. Rare cases have been recognized as late as 6 months of age.

Remarkably, this type of diabetes often responds well to sulfonylureas and insulin may not be necessary. More severe mutations in the KCNJ11 gene can cause early-onset diabetes which does not respond to the sulfonylurea drugs, as well as a syndrome of developmental delay and neurological features called the DEND syndrome. These forms of diabetes are very rare conditions, appearing in about 1/100,000 to 1/200,000 live births, and accounting for about 1/1000 of type 1 diabetes cases. Fewer than 5% of the cases assumed to exist have been diagnosed, and most diabetes clinics around the world are checking for KCNJ11 mutations in any persons who developed apparent insulin-dependent diabetes without the typical type 1 antibodies before 6 months of age. At least some of these people have been able to change from insulin to sulfonylurea pills after decades of injections.

### II. BROWN ADIPOSE TISSUE

In histology, adipose tissue or body fat or fat depot or just fat is loose connective tissue composed of adipocytes. It is technically composed of roughly only 80% fat; fat in its solitary state exists in the liver and muscles. Adipose tissue is derived from lipoblasts. Its main role is to store energy in the form of fat, although it also cushions and insulates the body. Obesity or being overweight in humans and most animals does not depend on body weight but on the amount of body fat—to be specific, adipose tissue. Two types of adipose tissue exist: white adipose tissue (WAT) and brown adipose tissue (BAT). Adipose tissue also serves as an important endocrine organ by producing hormones such as leptin, resistin, and the cytokine TNFα. The formation of adipose tissue appears to be controlled by the adipose gene.

In humans, adipose tissue is located beneath the skin (subcutaneous fat), around internal organs (visceral fat), in bone marrow (yellow bone marrow) and in breast tissue. Adipose tissue is found in specific locations, which are referred to as ‘adipose depots.’ Adipose tissue contains several cell types, with the highest percentage of cells being adipocytes, which contain fat droplets. Other cell types include fibroblasts, macrophages, and endothelial cells. Adipose tissue contains many small blood vessels. In the integumentary system, which includes the skin, it accumulates in the deepest level, the subcutaneous layer, providing insulation from heat and cold. Around organs, it provides protective padding. However, its main function is to be a reserve of lipids, which can be burned to meet the energy needs of the body. Adipose depots in different parts of the body have different biochemical profiles.

In mice, there are eight major adipose depots, four of which are within the abdominal cavity: The paired gonadal depots are attached to the uterus and ovaries in females and the epididymis and testes in males; the paired retroperitoneal depots are found along the dorsal wall of the abdomen, surrounding the kidney, and, when massive, extend into the pelvis. The mesenteric depot forms a glue-like web that supports the intestines, and the omental depot, which originates near the stomach and spleen, and, when massive, extends into the ventral abdomen. Both the mesenteric and omental depots incorporate much lymphoid tissue as lymph nodes and milky spots, respectively. The two superficial depots are the paired inguinal depots, which are found anterior to the upper segment of the hind limbs (underneath the skin) and the subscapular depots, paired medial mixtures of brown adipose tissue adjacent to regions of white adipose tissue, which are found under the skin between the dorsal crests of the scapulae. The layer of brown adipose tissue in this depot is often covered by a “frosting” of white adipose tissue; sometimes these two types of fat (brown and white) are hard to distinguish. The inguinal depots enclose the inguinal group of lymph nodes. Minor depots include the pericardial, which surrounds the heart, and the paired popliteal depots, between the major muscles behind the knees, each containing one large lymph node. Of all the depots in the mouse, the gonadal depots are the largest and the most easily dissected, comprising about 30% of dissectible fat.

In a severely obese person, excess adipose tissue hanging downward from the abdomen is referred to as a panniculus (or pannus). A panniculus complicates surgery of the morbidly obese. The panniculus may remain as a literal “apron of skin” if a severely obese person quickly loses large amounts of fat (a common result of gastric bypass surgery). This condition cannot be effectively corrected through diet and exercise alone, as the panniculus consists of adipocytes and other supporting cell types shrunken to their minimum volume and diameter. Reconstructive surgery is one method of treatment.

Visceral fat or abdominal fat also known as organ fat or intra-abdominal fat, is located inside the abdominal cavity, packed in between organs (stomach, liver, intestines, kidneys, etc.). Visceral fat is different than subcutaneous fat underneath the skin, and intramuscular fat interspersed in skeletal muscles. Fat in the lower body, as in thighs and buttocks, is subcutaneous, whereas fat in the abdomen is mostly visceral. Visceral fat is composed of several adipose depots including mesenteric, epididymal white adipose tissue (EWAT) and perirenal depots.

An excess of visceral fat is known as central obesity, or “belly fat,” in which the abdomen protrudes excessively. There is a strong correlation between central obesity and cardiovascular disease. Excess visceral fat is also linked to diabetes, insulin resistance inflammatory diseases, and other obesity-related diseases.

Female sex hormone causes fat to be stored in the buttocks, thighs, and hips in women. Men are more likely to have fat stored in the belly due to sex hormone differences. When women reach menopause and the estrogen produced by ovaries declines, fat migrates from their buttocks, hips and thighs to their waists; later fat is stored in the belly.

High intensity exercise effectively reduces total abdominal fat. At least 10 METs×hours per week in aerobic exercise is required for visceral fat reduction.

Most of the remaining non-visceral fat is found just below the skin in a region called the hypodermis. This subcutaneous fat is not related to many of the classic obesity-related pathologies, such as heart disease, cancer, and stroke, and there is even some evidence that it might be protective. The typically female (or gynecoid) pattern of body fat distribution around the hips, thighs, and buttocks, is subcutaneous fat, and therefore poses less of a health risk compared to visceral fat.

Like all other fat organs, subcutaneous fat is an active part of the endocrine system, secreting the hormones leptin and resistin. Free fatty acid is liberated from lipoproteins by lipoprotein lipase (LPL) and enters the adipocyte, where it is reassembled into triglycerides by esterifying it onto glycerol. Human fat tissue contains about 87% lipids. In humans, lipolysis is controlled through the balanced control of lipolytic B-adrenergic receptors and a2A-adrenergic receptor-mediated antilipolysis. Fat is not laid down when there is surplus calories available and stored passively until it is needed; rather it is constantly being stored in and released from the adipose tissue. Storage in the adipose tissue is catalysed by insulin, the activity of which is stimulated by high blood sugar

Fat cells have an important physiological role in maintaining triglyceride and free fatty acid levels, as well as determining insulin resistance. Abdominal fat has a different metabolic profile—being more prone to induce insulin resistance. This explains to a large degree why central obesity is a marker of impaired glucose tolerance and is an independent risk factor for cardiovascular disease (even in the absence of diabetes mellitus and hypertension). Studies of female monkeys discovered that individuals suffering from higher stress have higher levels of visceral fat in their bodies. This suggests a possible cause-and-effect link between the two, wherein stress promotes the accumulation of visceral fat, which in turn causes hormonal and metabolic changes that contribute to heart disease and other health problems.

Recent advances in biotechnology have allowed for the harvesting of adult stem cells from adipose tissue, allowing stimulation of tissue regrowth using a patient's own cells. In addition, it was reported that adipose-derived stem cells from both human and animals can be efficiently reprogrammed into induced pluripotent stem cells without the need for feeder cells. The use of a patient's own cells reduces the chance of tissue rejection and avoids the ethical issues associated with the use of human embryonic stem cells.

Adipose tissue is the greatest peripheral source of aromatase in both males and females contributing to the production of estradiol. Adipose derived hormones include Adiponectin, Resistin, Plasminogen activator inhibitor-1 (PAI-1), TNFα, IL-6, Leptin and Estradiol (E2). Adipose tissues also secrete a type of cytokines (cell-to-cell signalling proteins) called adipokines (adipocytokines) which play a role in obesity-associated complications.

A specialized form of adipose tissue in humans, most rodents and small mammals, and some hibernating animals, is brown fat or brown adipose tissue. It is located mainly around the neck and large blood vessels of the thorax. This specialised tissue can generate heat by “uncoupling” the respiratory chain of oxidative phosphorylation within mitochondria. The process of uncoupling means that, when protons transit down the electrochemical gradient across the inner mitochondrial membrane, the energy from this process is released as heat rather than being used to generate ATP. This thermogenic process may be vital in neonates exposed to the cold, who then require this thermogenesis to keep warm as they are unable to shiver, or take other actions to keep themselves warm.

Attempts to simulate this process pharmacologically have so far been unsuccessful (even lethal). Techniques to manipulate the differentiation of “brown fat” could become a mechanism for weight loss therapy in the future, encouraging the growth of tissue with this specialized metabolism without inducing it in other organs.

Until recently, it was thought that brown adipose tissue was primarily limited to infants in humans, but new evidence has now overturned that belief. Metabolically active tissue with temperature responses similar to brown adipose was first reported in the neck and trunk of some human adults in 2007, and the presence of brown adipose in human adults was later verified histologically in the same anatomical regions.

F. Pre-Diabetic Metabolic Syndrome

Metabolic Syndrome, also known as Syndrome X, has several tell-tale symptoms: excessive abdominal fat, high LDL and low HDL levels, high triglycerides and high blood pressure. These are all implicated in the development of Pre- and Type 2 Diabetes, and substantially increase chances of developing Cardiovascular Disease (CVD). A root cause of Metabolic Syndrome is Insulin Resistance. The latter condition increases the risk of developing Pre-Diabetes and Type 2 Diabetes, which may result in a heart attack or stroke. People with often Insulin Resistance-linked blood glucose levels that are higher than normal but not yet in the Type 2 Diabetes range have Pre-Diabetes. Doctors sometimes call this condition impaired fasting glucose (IFG) or impaired glucose tolerance (IGT), depending on the blood test used to diagnose it.

### III. TRANSPLANTATION

A. Allograft Transplants

In one aspect, the present invention involves the use of transplated brown adipose tissue in a surgical procedure. This is a relatively common procedure that has as variety of different utilities, but is particularly useful in the context of cosmetic and constructive surgery. For example, in secondary mammary reconstruction in irradiated patients, fat tissue can be used in conjuction with tissue expanders and prostheses. The results showed that fat grafting in addition to traditional tissue expander and implant breast reconstruction achieves better reconstructive outcomes with the creation of new subcutaneous tissue, accompanied by improved skin quality of the reconstructed breast without capsular contracture (Serra-Renom et al., 2010).

A common source of BAT is cadaver tissue, with the majority of tissue in normal adults being found in the head, neck and chest region. Another source of BAT is the waste product of liposuction, although the majority of fat removed in such procedures is white adipose tissue. Yet another option is the use of adipose-derived stem cells (ASCs). These cells possess multipotency in vivo, and thus are promising precursors for use in regenerative transplants. ASCs can be concentrated from adipose tissue by enzymatic digestion prior to transplantation. Adipose tissue is thought to be a promising source of stem cells because it can be harvested in relatively large quantities (100 mL to >1 L) using liposuction. U.S. Patent Publication 2010/0028310, which describes compositions for the transplant of adipose tissue and ASCs is incorporated by reference. Seale et al. (2011), also incorporated by reference, describes the selection of BAT precursors using Prdm 16 a marker for brown fat-like gene expression in WAT.

Although ASCs may be clinically used without cell expansion because of their large quantities, it is of great value to culture and expand ASCs safely and effectively without losing their multipotency for manipulation and further development of cell-based therapies. There have been reports indicating enhanced proliferation of human ASCs using specific culture media with supplements. For example, it has been shown that fibroblast growth factor (FGF)-2 is released by ASCs, enhances proliferation, and maintains the adipogenic potential of ASCs. FGF-1 and epidermal growth factor (EGF) have also been suggested to act as stimulators of both ASC proliferation and differentiation. Platelet-derived growth factor (PDGF)-BB, tumor necrosis factor (TNF)-α, and insulin-like growth factor (IGF)-1 are also shown to promote ASC proliferation, and the former two factors were suggested to have inhibiting effects on ASC differentiation. A prepared medium, called EGM-2, is often used to support the growth of endothelial cells. It contains 2% FBS and a cocktail of various growth factors including FGF-2, VEGF, IGF-1, and EGF. EGM-2 expanded ASCs very rapidly while preserving their multipotency for at least 2 weeks; the proliferative efficiency of EGM-2 was 105 times of that of DMEM in the first 2 weeks.

B. Autograft Stem Cell Transplants

As discussed above, ASCs possess multipotency in vivo, and thus are promising precursors for use in regenerative transplants. The added advantage of a stem cell transplant, particularly after in vitro expansion (also discussed above), would be reduced chance for adverse inflammatory reactions in the transplant recipient. Various other sources of stem cells include embryonic stem cells (ESCs), induced pluripotent stem cells (iPSCs) and hematopoietic stem cells. Methods for producing BAT and BAT stem cells from multipotent stems cells are described in U.S. Patent Publications 2011/0144009 and 2010/0150885, which are hereby incorporated by reference. In addition, brown adipose cells lines may be utilized.

C. Implantation

Following obtaining sufficient cells/tissues for transplantation, the cells/tissues are surgically implanted. Typically, the approach for implantation will be for subcutaneous delivery due to ease and simplicity. However, transplantation under the renal capsule, intramuscularly, and intra-abdominally (e.g., into viseceral fat such as omental and mesenteric fat depots).

### IV. ADJUNCT THERAPIES AND PROCEDURES

A. Insulin Therapy

In accordance with the present invention, it may prove advantageous to combine the methods disclosed herein with adjunct therapies or procedures to enhance the overall anti-diabetic effect. Such therapies and procedures are set forth in general, below. A skilled physician will be apprised of the most appropriate fashion in which these therapies and procedures may be employed.

The present invention, though designed to eliminate the need for other therapies, is contemplated to provide advantageous use with traditional insulin supplementation, but at lower levels, such as below 90%, below 80%, below 70%, below 60%, below 50%, below 40%, below 30%, below 20%, below 15%, 10-15%, below 10%, 5-10%, below 5%, 4%, 3%, 2% or 1% of the normal daily dosage of insulin. Normal daily dosage for TD1 is 30-60 units per day. Such therapies should be tailored specifically for the individual patient given their current clinical situation, and it is contemplated that a subject could be “weaned” down or off insulin therapy after commencing of leptin or leptin agonist provision. The following are general guidelines for typical a “monotherapy” using insulin supplementation by injection, and can be applied here, albeit in the context of the aforementioned reductions in total daily dosage.

Insulin can be injected in the thighs, abdomen, upper arms or gluteal region. In children, the thighs or the abdomen are preferred. These offer a large area for frequent site rotation and are easily accessible for self-injection. Insulin injected in the abdomen is absorbed rapidly while from the thigh it is absorbed more slowly. Hence, patients should not switch from one area to the other at random. The abdomen should be used for the time of the day when a short interval between injection and meal is desired (usually pre-breakfast when the child may be in a hurry to go to school) and the thigh when the patient can wait 30 minutes after injection for his meal (usually pre-dinner). Within the selected area systematic site rotation must be practiced so that not more than one or two injections a month are given at any single spot. If site rotation is not practiced, fatty lumps known as lipohypertrophy may develop at frequently injected sites. These lumps are cosmetically unacceptable and, what is more important, insulin absorption from these regions is highly erratic.

Before injecting insulin, the selected site should be cleaned with alcohol. Injecting before the spirit evaporates can prove to be quite painful. The syringe is held like a pen in one hand, pinching up the skin between the thumb and index finger of the other hand, and inserting the needle through the skin at an angle of 45-90° to the surface. The piston is pushed down to inject insulin into the subcutaneous space (the space between the skin and muscle), then one waits for a few seconds after which release the pinched up skin before withdrawing the needle. The injection site should not be massaged.

For day-to-day management of diabetes, a combination of short acting and intermediate acting insulin is used. Some children in the first year after onset of diabetes may remain well controlled on a single injection of insulin each day. However, most diabetic children will require 2, 3 or even 4 shots of insulin a day for good control. A doctor should decide which regimen is best suited.

One injection regimen: A single injection comprising a mix of short acting and intermediate acting insulin (mixed in the same syringe) in 1:3 or 1:4 proportion is taken 20 to 30 minutes before breakfast. The usual total starting dose is 0.5 to 1.0 units/kg body weight per day. This regimen has three disadvantages: (1) all meals must be consumed at fixed times; (2) since the entire quantity of insulin is given at one time, a single large peak of insulin action is seen during the late and early evening hours making one prone to hyopglycemia at this time; (3) as the action of intermediate acting insulin rarely lasts beyond 16-18 hours, the patient's body remains underinsulinized during the early morning hours, the period during which insulin requirement in the body is actually the highest.

Two-injection regimen: This regimen is fairly popular. Two shots of insulin are taken—one before breakfast (⅔ of the total dose) and the other before dinner (⅓ of the total dose). Each is a combination of short acting and intermediate acting insulin in the ratio of 1:2 or 1:3 for the morning dose, and 1:2 or 1:1 for the evening dose. With this regimen the disadvantages of the single injection regimen are partly rectified. Some flexibility is possible for the evening meal. Further, as the total days' insulin is split, single large peaks of insulin action do not occur hence risk of hypoglycemia is reduced and one remains more or less evenly insulinized throughout the day. On this regimen, if the pre-breakfast blood glucose is high, while the 3 a.m. level is low, then the evening dose may need to be split so as to provide short acting insulin before dinner and intermediate acting insulin at bedtime.

Multi-dose insulin regimens: The body normally produces insulin in a basal-bolus mariner, i.e., there is a constant basal secretion unrelated to meal intake and superimposed on this there is bolus insulin release in response to each meal. Multi-dose insulin regimens were devised to mimic this physiological pattern of insulin production. Short acting insulin is taken before each major meal (breakfast, lunch and dinner) to provide “bolus insulin” and intermediate acting insulin is administered once or twice a day for “basal insulin.” Usually bolus insulin comprises 60% of the total dose and basal insulin makes up the remaining 40%. With this regimen you have a lot of flexibility. Both the timing as well as the quantity of each meal can be altered as desired by making appropriate alterations in the bolus insulin doses. To take maximum advantage of this regimen, one should learn “carbohydrate counting” and work out carbohydrate:insulin ratio—the number of grams of carbohydrate for which the body needs 1 unit of insulin.

B. Leptin Therapy

Leptin (Greek leptos meaning thin) is a 16 kDa protein hormone that plays a key role in regulating energy intake and energy expenditure, including appetite and metabolism. Leptin is one of the most important adipose derived hormones.

Thus, circulating leptin levels give the brain input regarding energy storage so it can regulate appetite and metabolism. Leptin works by inhibiting the activity of neurons that contain neuropeptide Y (NPY) and agouti-related peptide (AgRP), and by increasing the activity of neurons expressing α-melanocyte-stimulating hormone (α-MSH). The NPY neurons are a key element in the regulation of appetite; small doses of NPY injected into the brains of experimental animals stimulates feeding, while selective destruction of the NPY neurons in mice causes them to become anorexic. Conversely, α-MSH is an important mediator of satiety, and differences in the gene for the receptor at which α-MSH acts in the brain are linked to obesity in humans.

Once leptin has bound to the Ob-Rb receptor, it activates the Stat3, which is phosphorylated and travels to the nucleus to, presumably, effect changes in gene expression. One of the main effects on gene expression is the down-regulation of the expression of endocannabinoids, responsible for increasing appetite. There are other intracellular pathways activated by leptin, but less is known about how they function in this system. In response to leptin, receptor neurons have been shown to remodel themselves, changing the number and types of synapses that fire onto them.

Although leptin is a circulating signal that reduces appetite, in general, obese people have an unusually high circulating concentration of leptin. These people are said to be resistant to the effects of leptin, in much the same way that people with type 2 diabetes are resistant to the effects of insulin. The high sustained concentrations of leptin from the enlarged adipose stores result in leptin desensitization. The pathway of leptin control in obese people might be flawed at some point so the body doesn't adequately receive the satiety feeling subsequently to eating.

The body's fat cells, under normal conditions, are responsible for the constant production and release of leptin. This can also be produced by the placenta. Leptin levels rise during pregnancy and fall after parturition (childbirth). Leptin is also expressed in fetal membranes and the uterine tissue. Uterine contractions are inhibited by leptin.

Leptin therapies for diabetes are described in detail in WO 2010/022262, which is hereby incorporated by reference.

C. Monitoring Glucose Levels

Any person suffering from diabetes will be very familiar with the need to regularly measure blood glucose levels. Blood glucose level is the amount of glucose, or sugar, in the blood. It is also is referred to as “serum glucose level.” Normally, blood glucose levels stay within fairly narrow limits throughout the day (4 to 8 mmol/l), but are often higher after meals and usually lowest in the morning. Unfortunately, when a person has diabetes, their blood glucose level sometimes moves outside these limits. Thus, much of a diabetic's challenge is to When one suffers from diabetes, it is important that glucose level be as near normal as possible. Stable blood glucose significantly reduces the risk of developing late-stage diabetic complications, which start to appear 10 to 15 years after diagnosis with type 1 diabetes, and often less than 10 years after diagnosis with type 2 diabetes.

Blood glucose levels can be measured very simply and quickly with a home blood glucose level testing kit, consisting of a measuring device itself and a test strip. To check blood glucose level, a small amount of blood is placed on the test strip, which is then placed into the device. After about 30 seconds, the device displays the blood glucose level. The best way to take a blood sample is by pricking the finger with a lancet. Ideal values are (a) 4 to 7 mmol/l before meals, (b) less than 10 mmol/l one-and-a-half hours after meals; and (c) around 8 mmol/l at bedtime.

People who have type 1 diabetes should measure their blood glucose level once a day, either in the morning before breakfast or at bedtime. In addition, a 24-hour profile should be performed a couple of times a week (measuring blood glucose levels before each meal and before bed). People who have type 2 diabetes and are being treated with insulin should also follow the schedule above. People who have type 2 diabetes and who are being treated with tablets or a special diet should measure their blood glucose levels once or twice a week, either before meals or one-and-a-half hours after a meal. They should also perform a 24-hour profile once or twice a month.

The main advantage for measuring blood glucose levels of insulin-treated diabetics in the morning is that adjusted amounts of insulin can be taken if the blood glucose level is high or low, thereby reducing the risk of developing late-stage diabetic complications. Similarly, the blood glucose level at bedtime should be between 7 and 10 mmol/l. If blood glucose is very low or very high at bedtime, there may be a need to adjust food intake or insulin dose. Blood glucose should also be measured any time the patient does not feel well, or think blood glucose is either too high or too low. People who have type 1 diabetes with a high level of glucose in their blood (more than 20 mmol/l), in addition to sugar traces in the urine, should check for ketone bodies in their urine, using a urine strip. If ketone bodies are present, it is a warning signal that they either have, or may develop, diabetic acidosis.

D. Amylin

Amylin, or Islet Amyloid Polypeptide (IAPP), is a 37-residue peptide hormone secreted by pancreatic β-cells at the same time as insulin (in a roughly 1:100 amylin:insulin ratio). There appear to be at least three distinct receptor complexes that bind with high affinity to amylin. All three complexes contain the calcitonin receptor at the core, plus one of three receptor activity-modifying proteins, RAMP1, RAMP2, or RAMP3. Amylin is degraded in part by insulin-degrading enzyme.

Amylin is commonly found in pancreatic islets of patients suffering diabetes mellitus type 2, or harboring an insulinoma. While the association of amylin with the development of type 2 diabetes has been known for some time, a direct causative role for amylin has been harder to establish. Recent results suggest that amylin, like the related beta-amyloid (Abeta) associated with Alzheimer's disease, can induce apoptotic cell-death in insulin-producing beta cells, an effect that may be relevant to the development of type 2 diabetes. Finally, a recent study reported a synergistic effect for weight loss with leptin and amylin coadministration in diet-induced obese rats by restoring hypothalamic sensitivity to leptin.

Amylin functions as part of the endocrine pancreas and contributes to glycemic control. The peptide is secreted from the pancreas into the blood circulation and eventually excreted by the kidneys. Amylin's metabolic function is now somewhat well characterized as an inhibitor of the appearance of nutrient, especially glucose, in the plasma. It thus functions as a synergistic partner to insulin, with which it is cosecreted from pancreatic beta cells in response to meals. The overall effect to slow the rate of appearance (Ra) from the meal is mediated via a coordinate reduction of food intake, slowing of gastric emptying, inhibition of digestive secretion. Appearance of new glucose is slowed by inhibiting secretion of the gluconeogenic hormone glucagon. These actions, which are mostly mediated via a glucose-sensitive part of the brain stem, the area postrema, may be over-ridden during hypoglycemia. They collectively reduce the total insulin demand. Amylin also acts in bone metabolism, along with the related peptides calcitonin and calcitonin gene related peptide.

Rodent amylin knockouts are known to fail to achieve the normal anorexia following food consumption. Because it is an amidated peptide, like many neuropeptides, it is believed to be responsible for the anorectic effect.

The human form of amylin has the amino acid sequence KCNTATCATQRLANFLVHSSNNFGAILSSTNVGSNTY, with a disulfide bridge between cysteine residues 2 and 7. Both the amidated C-terminus and the disulfide bridge are necessary for the full biological activity of amylin. Amylin is capable of forming amyloid fibrils in vitro. Within the fibrillization reaction, the early prefibrillar structures are extremely toxic to beta-cell and insuloma cell cultures. Later amyloid fiber structures also seem to have some cytotoxic effect on cell cultures. Studies have shown that fibrils are the end product and not necessarily the most toxic form of amyloid proteins/peptides in general. A non-fibril forming peptide (1-19 residues of human amylin) is toxic like the full-length peptide but the respective segment of rat amylin is not. It was also demonstrated by solid-state NMR spectroscopy that the fragment 20-29 of the human-amylin fragments membranes. Rats and mice have six substitutions (three of which are proline substitions at positions 25, 28 and 29) that are believed to prevent the formation of amyloid fibrils. Rat IAPP is nontoxic to beta-cells, even when overexpressed.

A synthetic analog of human amylin with proline substitutions in positions 25, 26 and 29, or pramlintide (brand name Symlin®, Amylin Pharmaceuticals), was recently approved for adult use in patients with both diabetes mellitus type 1 and diabetes mellitus type 2. Insulin and pramlintide, injected separately but both before a meal, work together to control the post-prandial glucose excursion.

E. GLP-1 Analogs

Glucagon-like peptide-1 (GLP-1) is derived from the transcription product of the proglucagon gene. The major source of GLP-1 in the body is the intestinal L cell that secretes GLP-1 as a gut hormone. The biologically active forms of GLP-1 are: GLP-1-(7-37) and GLP-1-(7-36)NH2. Those peptides result from selective cleavage of the proglucagon molecule.

GLP-1 secretion by ileal L cells is dependent on the presence of nutrients in the lumen of the small intestine. The secretagogues (agents that cause or stimulate secretion) of this hormone include major nutrients like carbohydrate, protein and lipid. Once in the circulation, GLP-1 has a half life of less than 2 minutes, due to rapid degradation by the enzyme dipeptidyl peptidase-4. It is a potent antihyperglycemic hormone, inducing glucose-dependent stimulation of insulin secretion while suppressing glucagon secretion. Such glucose-dependent action is particularly attractive because when the plasma glucose concentration is in the normal fasting range, GLP-1 no longer stimulates insulin to cause hypoglycemia. GLP-1 appears to restore the glucose sensitivity of pancreatic β-cells, with the mechanism possibly involving the increased expression of GLUT2 and glucokinase. GLP-1 is also known to inhibit pancreatic β-cell apoptosis and stimulate the proliferation and differentiation of insulin-secreting β-cells. In addition, GLP-1 inhibits gastric secretion and motility. This delays and protracts carbohydrate absorption and contributes to a satiating effect.

Glucagon-like peptide-1 analogs are a new class of drug for treatment of type 2 diabetes. One of their advantages is that they have a lower risk of causing hypoglycemia. Exenatide (Byetta®; Amylin Pharmaceuticals; Eli Lilly & Co.) was approved in the U.S. on Apr. 28, 2005 for the treatment of diabetes mellitus type 2. It is administered as a subcutaneous injection (under the skin) of the abdomen, thigh, or arm, 30 to 60 minutes before the first and last meal of the day. Exenatide (39 aa) is a synthetic version of exendin-4, a hormone found in the saliva of the Gila monster. It displays biological properties similar to human glucagon-like peptide-1 (GLP-1). Exenatide enhances glucose-dependent insulin secretion by the pancreatic beta-cell, suppresses inappropriately elevated glucagon secretion, and slows gastric emptying, although the mechanism of action is still under study.

Liraglutide, marketed under the brand name Victoza®, is a long-acting glucagon-like peptide-1 (GLP-1) analog developed by Novo Nordisk for the treatment of type 2 diabetes. The product was approved on in the U.S. on Jan. 25, 2010. Liraglutide is an acylated human Glucagon-Like Peptide-1 (GLP-1) receptor agonist with 97% amino acid sequence homology to endogenous human GLP-1(7-37). Liraglutide is given once-daily. GLP-1, in its natural form, is short-lived in the body (the half-life after subcutaneous injection is approximately one hour), so it is not very useful as a therapeutic agent. However, liraglutide has a half-life after subcutaneous injection of 11-15 hours, making it suitable for once-daily dosing (less frequent than the currently approved Byetta® form of exenatide, which is twice daily, but considerably more frequent than the once weekly Bydureon® form of exenatide that is awaiting a decision from the FDA regarding marketing approval). The prolonged action of liraglutide is achieved by attaching a fatty acid molecule at one position of the GLP-1 molecule, enabling it to bind to albumin within the subcutaneous tissue and bloodstream. The active GLP-1 is then released from albumin at a slow, consistent rate. Binding with albumin also results in slower degradation and reduced elimination of liraglutide from the circulation by the kidneys compared to GLP-1.

Albiglutide and taspoglutide are currently under evaluation.

F. Sulfonylureas

Sulfonylurea (derivatives are a class of antidiabetic drugs that are used in the management of diabetes mellitus type 2. They act by increasing insulin release from the beta cells in the pancreas. All sulfonylureas contain a central S-phenylsulfonylurea structure with p-substitution on the phenyl ring and various groups terminating the urea N′ end group. Sulfonylureas bind to an ATP-dependent K+(KATP) channel on the cell membrane of pancreatic beta cells. This inhibits a tonic, hyperpolarizing efflux of potassium, thus causing the electric potential over the membrane to become more positive. This depolarization opens voltage-gated Ca²⁺ channels. The rise in intracellular calcium leads to increased fusion of insulin granulae with the cell membrane, and therefore increased secretion of (pro)insulin. There is some evidence that sulfonylureas also sensitize β-cells to glucose, that they limit glucose production in the liver, that they decrease lipolysis (breakdown and release of fatty acids by adipose tissue) and decrease clearance of insulin by the liver.

Various sulfonylureas have different pharmacokinetics. The choice depends on the propensity of the patient to develop hypoglycemia—long-acting sulfonylureas with active metabolites can induce hypoglycemia. They can, however, help achieve glycemic control when tolerated by the patient. The shorter-acting agents may not control blood sugar levels adequately. Due to varying half-life, some drugs have to be taken twice (e.g., tolbutamide) or three times a day rather than once (e.g., glimepiride). The short-acting agents may have to be taken about 30 minutes before the meal, to ascertain maximum efficacy when the food leads to increased blood glucose levels.

Some sulfonylureas are metabolised by liver metabolic enzymes (cytochrome P450) and inducers of this enzyme system (such as the antibiotic rifampicin) can therefore increase the clearance of sulfonylureas. In addition, because some sulfonylureas are bound to plasma proteins, use of drugs that also bind to plasma proteins can release the sulfonylureas from their binding places, leading to increased clearance.

Sulfonylureas are used almost exclusively in diabetes mellitus type 2. Sulfonylureas are ineffective where there is absolute deficiency of insulin production such as in type 1 diabetes or post-pancreatectomy. Although for many years sulfonylureas were the first drugs to be used in new cases of diabetes, in the 1990s it was discovered that obese patients might benefit more from metformin. In about 10% of patients, sulfonylureas alone are ineffective in controlling blood glucose levels. Addition of metformin or a thiazolidinedione may be necessary, or (ultimately) insulin. Triple therapy of sulfonylureas, a biguanide (metformin) and a thiazolidinedione is generally discouraged, but some doctors prefer this combination over resorting to insulin.

Sulfonylureas, as opposed to metformin, the thiazolidinediones, exenatide, symlin and other newer treatment agents may induce hypoglycemia as a result of excesses in insulin production and release. This typically occurs if the dose is too high, and the patient is fasting. Some people attempt to change eating habits to prevent this, however it can be counter productive. Like insulin, sulfonylureas can induce weight gain, mainly as a result their effect to increase insulin levels and thus utilization of glucose and other metabolic fuels. Other side-effects are: abdominal upset, headache and hypersensitivity reactions.

Sulfonylureas are potentially teratogenic and cannot be used in pregnancy or in patients who may become pregnant. Impairment of liver or kidney function increase the risk of hypoglycemia, and are contraindications. As other anti-diabetic drugs cannot be used either under these circumstances, insulin therapy is typically recommended during pregnancy and in hepatic and renal failure, although some of the newer agents offer potentially better options.

G. Pharmaceutical Compositions and Routes of Delivery

Where clinical applications are contemplated, it will be necessary to prepare pharmaceutical compositions in a form appropriate for administration to a subject. The compositions will generally be prepared essentially free of pyrogens, as well as other impurities that could be harmful to humans or animals. One will generally desire to employ appropriate salts and buffers to render stable cells suitable for introduction into a patient. Aqueous compositions of the present invention comprise an effective amount of stable cells dispersed in a pharmaceutically acceptable carrier or aqueous medium, and preferably encapsulated.

The phrase “pharmaceutically or pharmacologically acceptable” refer to compositions that do not produce adverse, allergic, or other untoward reactions when administered to an animal or a human. As used herein, “pharmaceutically acceptable carrier” includes any and all solvents, dispersion media, antibacterial and antifungal agents, isotonic and absorption delaying agents and the like. As used herein, this term is particularly intended to include biocompatible implantable devices and encapsulated cell populations. The use of such media and agents for pharmaceutically active substances is well known in the art. Except insofar as any conventional media or agent is incompatible with the compositions of the present invention, its use in therapeutic compositions is contemplated. Supplementary active ingredients also can be incorporated into the compositions.

Under ordinary conditions of storage and use, the cell preparations may further contain a preservative to prevent growth of microorganisms. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

The compositions will advantageously be administered by injection, including intravenously, intradermally, intraarterially, intraperitoneally, intracranially, intraarticularly, intraprostaticaly, intrapleurally, intramuscularly, intrahepatically, subcutaneously, or by other method or any combination of the forgoing as would be known to one of ordinary skill in the art.

As will be recognized by those in the field, a “therapeutically effective amount” refers to an mount of such that, when provided to a subject in accordance with the disclosed and claimed methods effects one of the following biological activities: treats type I diabetes; restores normoglycemia; reduces, suppresses, attenuates, or inhibits hyperglucogonemia or a condition associated with hyperglucogonemia; and increases adiponectin, leptin and/or visfatin; in a subject diagnosed with or otherwise having type 1 diabetes. In certain embodiments, such therapeutically effective amount effects such an activity in a subject that is essentially devoid of endogenous insulin. As understood in the art, such therapeutically effective amount will vary with many factors including the age and weight of the patient, the patient's physical condition, the condition to be treated, and other factors.

The term “unit dose” refers to a physically discrete unit suitable for use in a subject, each unit containing a predetermined quantity of the composition calculated to produce the desired response in association with its administration, i.e., the appropriate route and treatment regimen. The quantity to be administered, both according to number of treatments and unit dose, depends on the subject to be treated, the state of the subject, and the protection desired. Precise amounts of the therapeutic composition also depend on the judgment of the practitioner and are peculiar to each individual.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Research Design and Methods.

Embryonic BAT was transplanted in the subcutaneous space of STZ-diabetic recipients. Recipient mice included C57BL/6J mice, and NCRNU-M-M nude mice. Donor adipose tissue came from E16-E17 C57BL/6J embryos. Weight was recorded and basal fed blood samples were collected at regular intervals from mice that received BAT transplants as well as normal non-diabetic control mice and untreated diabetic control mice. Intra-peritoneal glucose tolerance tests were performed before BAT transplants and at monthly intervals after BAT transplants. Metabolic parameters such as blood glucose; insulin response to glucose and arginine; and plasma levels of adiponectin, leptin, glucagon and IGF-1 were measured from transplant and control groups at regular intervals. Three months after BAT transplant, mice were subjected to an additional glucose tolerance test in the presence of 5961, an inhibitor of insulin receptor. One insulin tolerance test was also performed between 2 and 3 months post-transplant. BAT transplant mice were euthanized at different time points after 3 months and tissues were collected. Pancreata were tested for insulin content by immunohistochemistry and radioimmunoassay (RIA), and adipose tissue at and around the transplant site was examined for UCP-1, IGF-1, and signs of inflammation by immunohistochemistry.

Animals.

Recipients were immune-deficient NCRNU-M-M nude mice (Taconic), and immune-competent C57BL/6J mice (Jackson Labs) rendered diabetic with streptozotocin (125 mg/Kg dissolved in ice-cold Na⁺ citrate buffer at pH 4.5; injected IP; repeated biweekly until diabetes induced). All recipients were 3-6 months of age at the time of transplants. Donor embryonic BAT was obtained from C57BL/6J embryos at gestational age E16.5-E17.5. Parents were purchased from Jackson laboratories and maintained in the Vanderbilt animal care facility. Animals were fed standard laboratory chow, and cared for according to the guidelines of the Vanderbilt Institutional Animal Care and Use Committee.

Isolation of Donor Tissue.

Pregnant females are anesthetized with ketamine/xylazine (110/10 mg/Kg) IP. A bilateral sub costal incision is made and extended by a midline transverse incision to expose the abdominal cavity. Uterine horns are exposed one at a time. Starting near the ovary, a longitudinal incision is made along the uterine horn. Embryos are removed and placed in sterile ice cold saline. The mouse is immediately killed by cervical dislocation. The embryos are rapidly dissected with Dumont forceps, the embryonic BAT from the inter-scapular region removed and placed in sterile ice-cold saline, and transplanted into recipients as quickly as possible.

Transplantation.

Freshly-isolated embryonic BAT was transplanted into diabetic NCRNU nude mice or C57BL/6J mice, underneath the skin of the dorsal body surface. Through a small (1-2 mm) incision, a subcutaneous pocket is made by blunt dissection. Donor tissue is introduced into the pocket with Dumont forceps and pushed in with blunt-ended micro spatula. The incision is closed by gentle pressure with hemostats without sutures. 4-6 lobules of embryonic BAT were introduced into each recipient. Surgeries were performed under general anesthesia with ketamine/xylazine (110/10 mg/Kg) IP, and postoperative analgesia was provided with buprenorphine 0.1 mg/Kg/day SQ as necessary.

Metabolic Parameters.

Streptozotocin-treated mice with fasting blood glucose levels over 300 mg/dl are selected as transplant candidates. Blood samples are collected from a tail-snip under isolfurane/oxygen anesthesia, for measurement of glucose, insulin and other hormones. Intraperitoneal glucose tolerance tests (IPGTT) are performed a week before the transplant, then every month after the transplant until euthanasia for tissue collection. IPGTT involves blood collection from 6-hour-fasted mice prior to (0 min) and 15, 30, 60 and 120 min after intraperitoneal injection of sterile glucose (Sigma) (2 g/Kg bw) under isofluorane/oxygen anesthesia. Basal non-fasting blood samples were also collected every two weeks when possible. Plasma samples were analyzed for insulin, adiponectin, leptin, glucagon and IGF-1. At optimum euglycemia, usually around 3 months after transplant, each mouse also underwent an insulin tolerance test and an IPGTT in the presence of S-961, an insulin-receptor antagonist. During these tests, an initial glucose measurement was done at −10 min immediately followed by IP injection of S-961, and the remaining blood collections proceeded as usual after injection of glucose at 0 min.

Post-Mortem Tissue Collection.

Mice were euthanized 2-6 months after transplantation, and adipose tissue and pancreas were harvested for histology and insulin measurements. The whole pancreas and the WAT from the subcutaneous space of the dorsal body surface were collected from BAT transplant groups as well as normal and diabetic control groups. Tissues were blotted to remove moisture, weighed, preserved in 4% paraformaldehyde and washed in PBS for histological analysis.

Histology.

Histological sections of pancreata were analyzed by immunostaining for insulin and CD34. To verify the inflammatory status of adipose tissue, histological sections were immunostained with and TNFα and IL6. To monitor the progress of BAT transplant, adipose tissues sections encompassing the transplant site were harvested at 2, 3, and 6 months and immunostained for UCP-1 and IGF-1.

Pancreatic Insulin Content.

Freshly harvested pacreata were homogenized in acid-ethanol and placed on a shaker at 4° C. for 48 hours. Tissue extracts were centrifuged for 30 min at 4° C. at 2500 rpm, supernatants collected, and analyzed for insulin with RIA.

### Example 2

### Results

BAT Transplants Promote Weight Gain and Euglycemia.

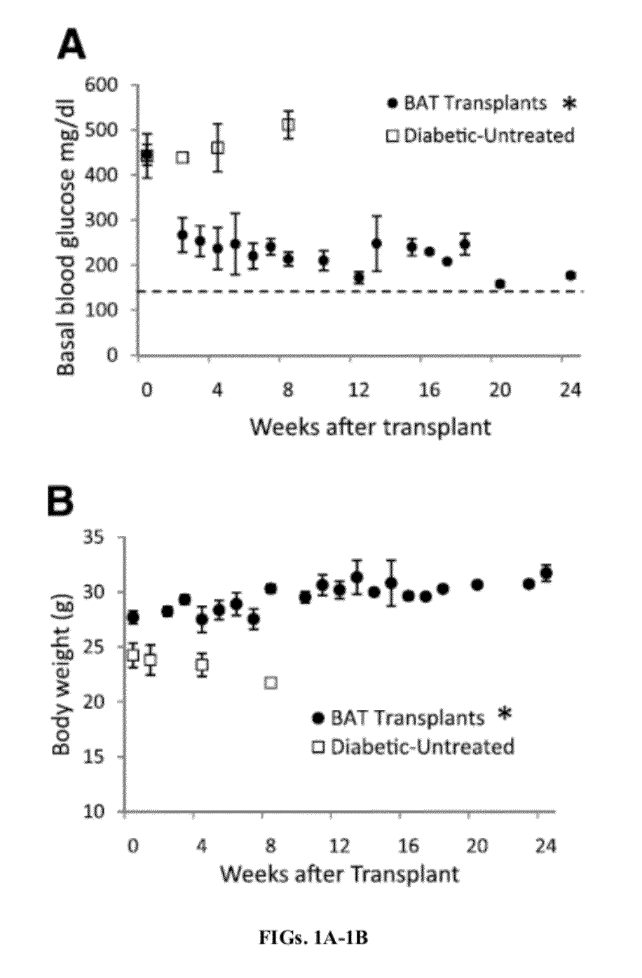

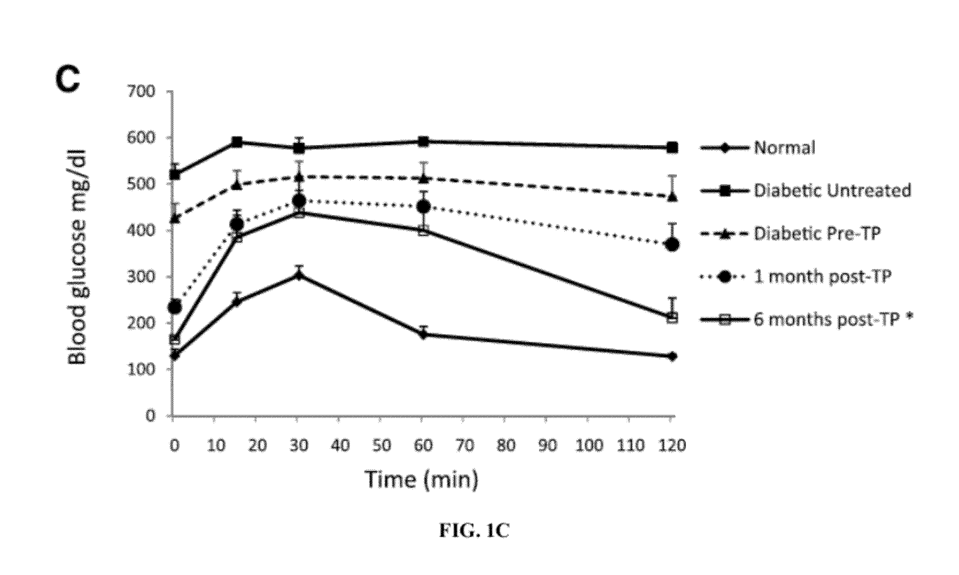

STZ-treated diabetic C57BL/6J mice receiving embryonic BAT transplants show marked improvement of glucose homeostasis (FIGS. 1A-D). Successful BAT transplants are defined as those diabetic mice whose basal blood glucose levels decreased by at least 200 mg/dl within 8 weeks of receiving a BAT transplant. FIGS. 1A-5B show the major results for immune-competent C57BL/6J mice, while the parallel findings in immune-deficient NCRNU nude mice (as well as additional data for C57BL/6J mice) are shown in the supplemental figures. Nine out of 14 transplants were successful (FIG. 1A), resulting in euglycemia and reversal of clinical signs of diabetes. These mice exhibit gradual weight gain, as opposed to the weight loss seen in the untreated diabetic control groups (FIG. 1B). This weight gain is partially attributable to the replenishment and expansion of subcutaneous white adipose tissue. Animals in the BAT transplant group have increased subcutaneous white adipose tissue compared to all other groups, both in total and as percent of body weight (Table 1). Weight gain and blood glucose normalization was accompanied by reversal of clinical signs of diabetes such as polyuria, polydipsia and polyphagia.

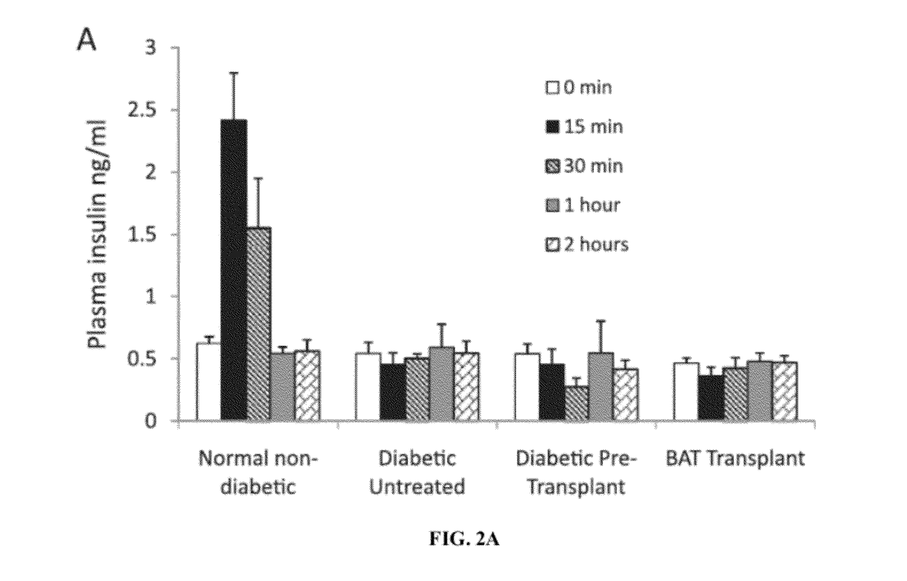

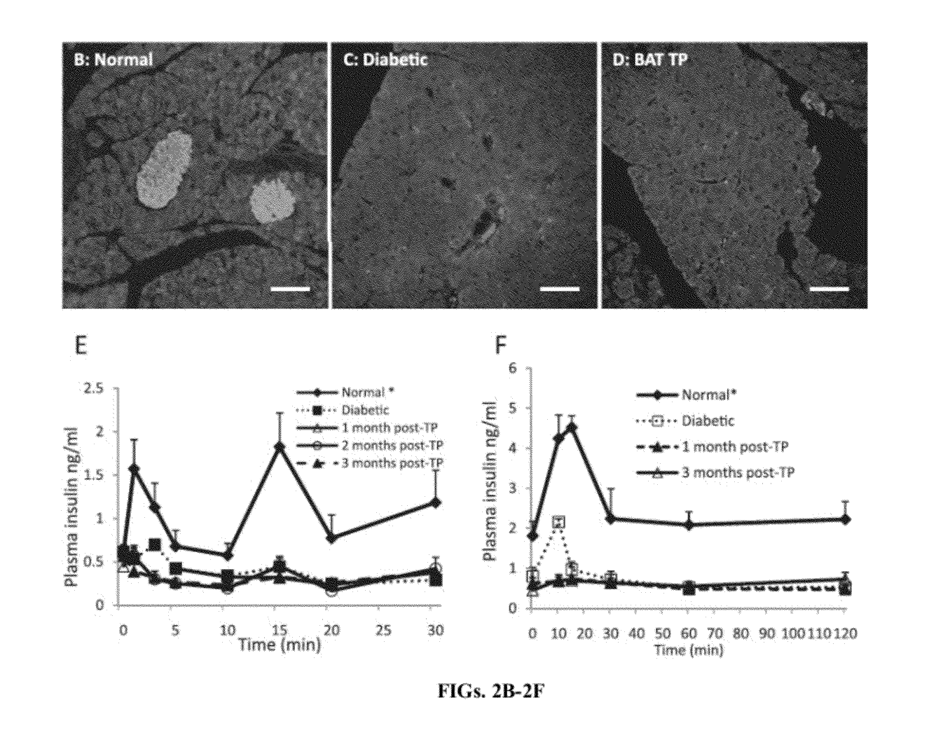

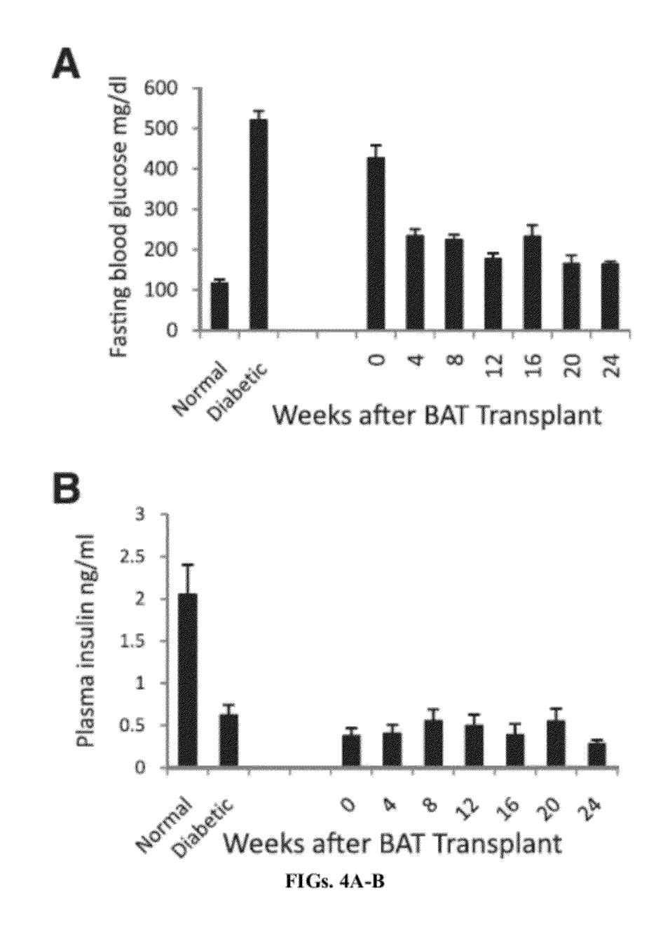

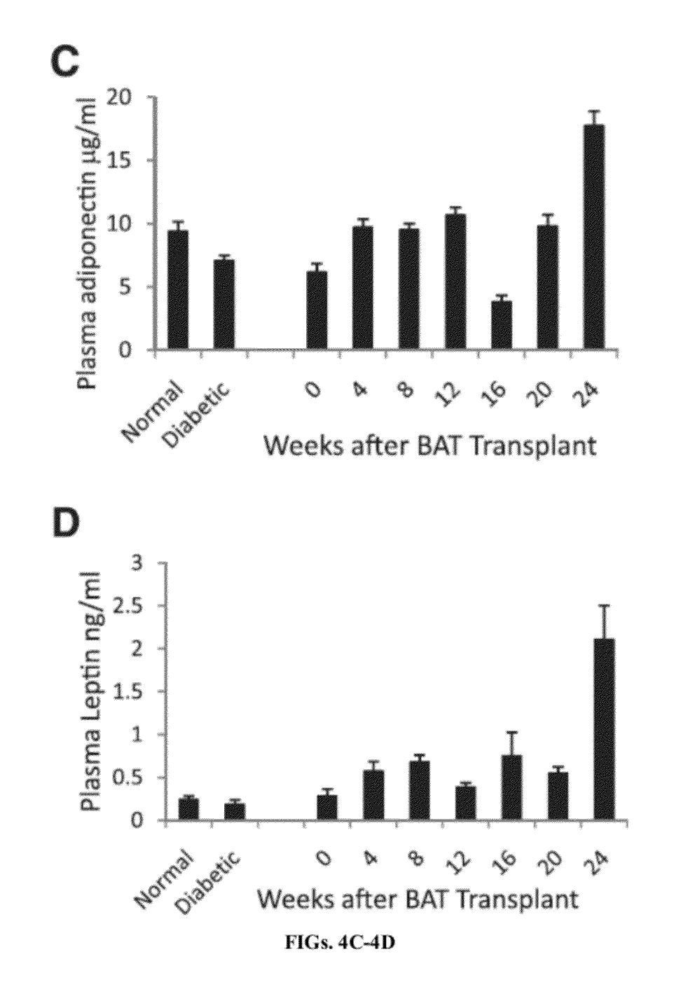

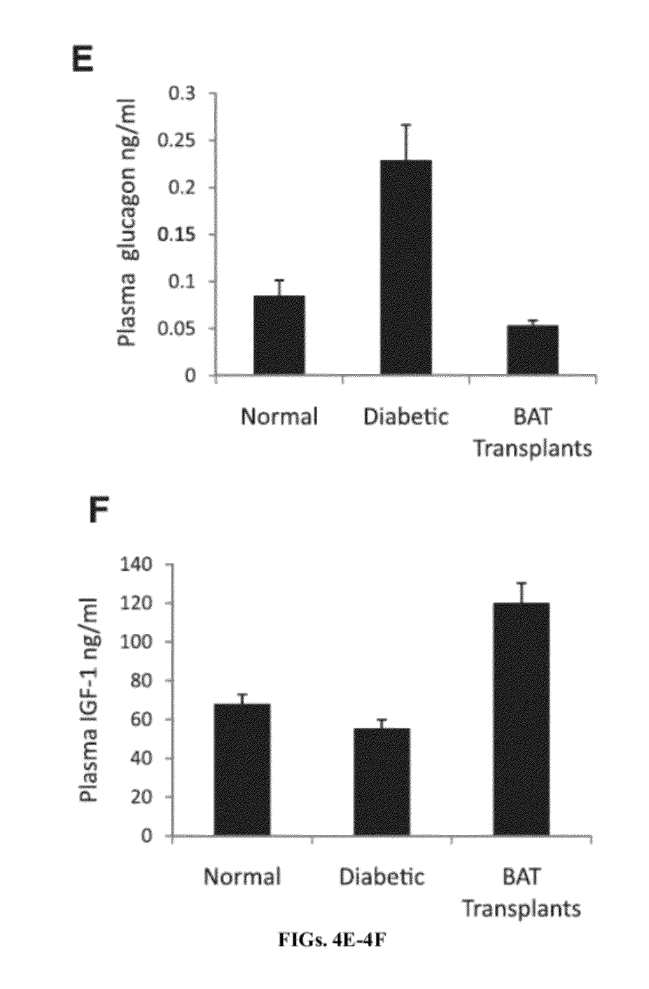

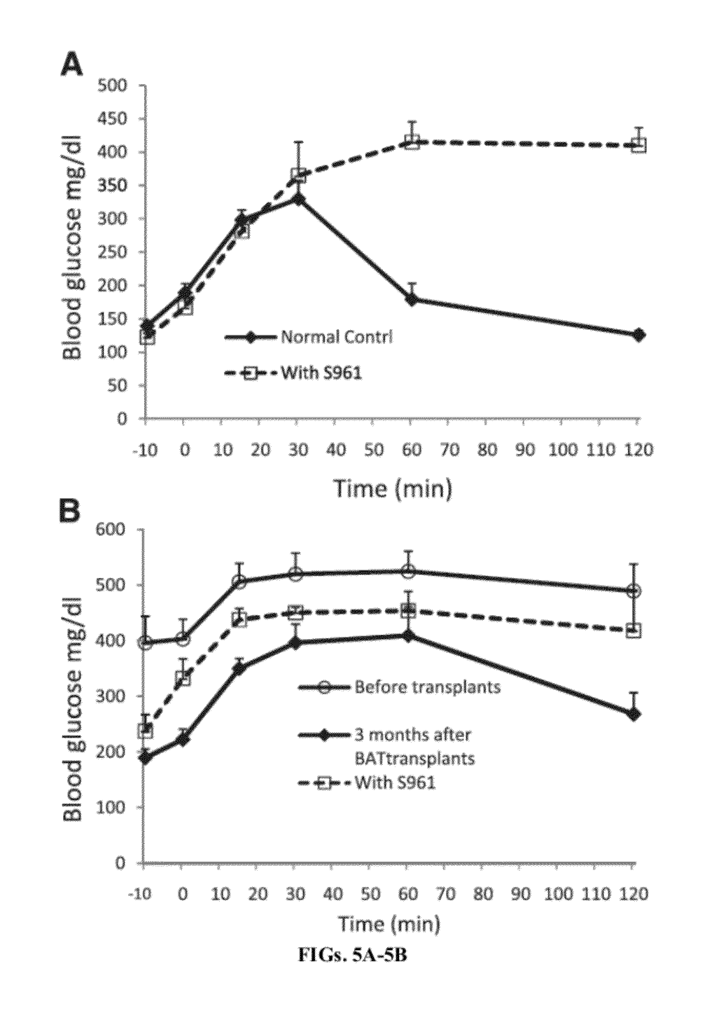

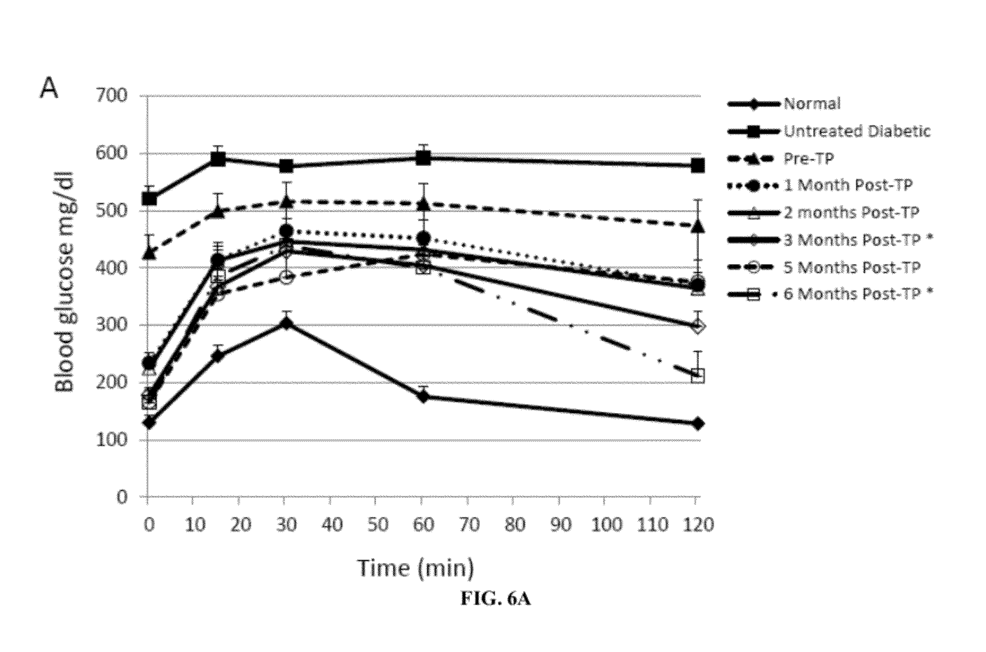

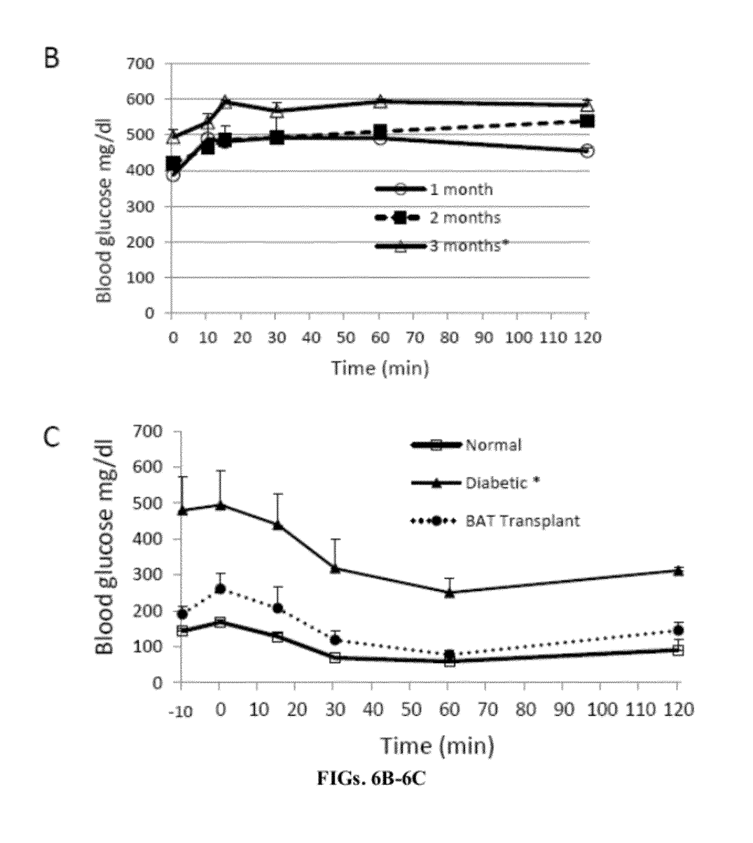

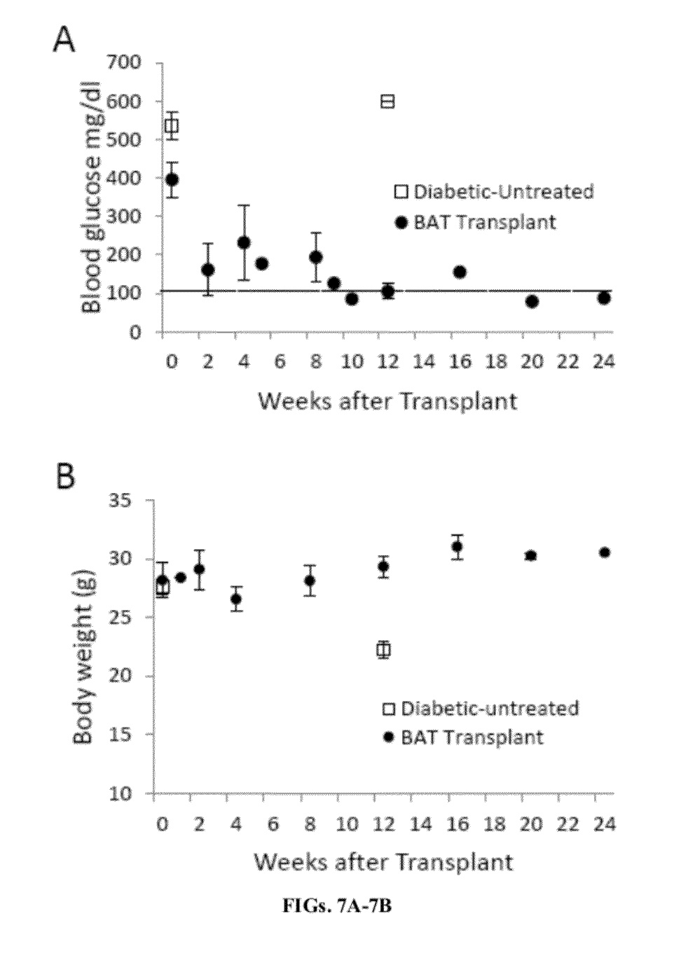

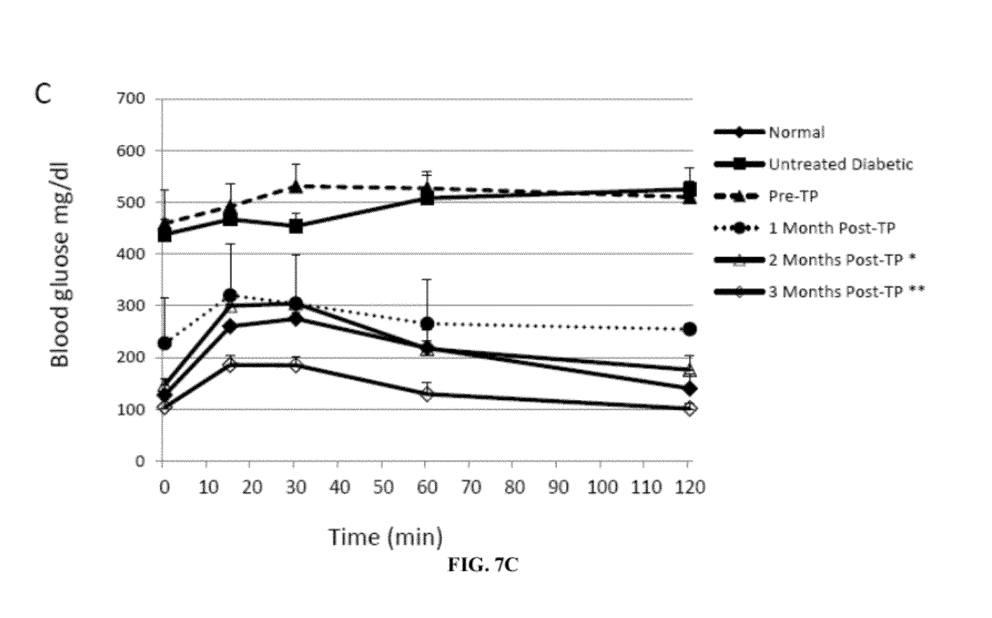

Prior to transplant, the diabetic mice exhibit basal blood glucose levels of 449±22 mg/dl. Basal glucose levels decrease sharply and significantly within 2 weeks, dropping to 267±38 mg/dl (FIG. 1A). These levels continue to fall until euthanasia at different endpoints between 3-6 months post-transplant. The BAT transplants result in improved glucose tolerance within 1 month of the transplant, and this tolerance becomes progressively closer to normal throughout the next six months (FIG. 1C & FIG. 6A). This is in sharp contrast to untreated diabetic control mice, whose glucose tolerance progressively deteriorates with time (FIG. 6B). There are no differences in plasma insulin levels measured in the BAT transplant mice and the STZ-treated diabetic control animals (FIGS. 2A and 4B). Insulin tolerance in successful BAT transplant mice at 2 or 3 months is not significantly different from non-diabetic control animals, and both are strikingly different than what is seen in STZ-treated diabetic mice (FIG. 6B). In the six BAT transplant mice that were maintained over 3 months, there was a transient episode of hyperglycemia occurring between 3-5 months, with impaired glucose tolerance (FIGS. 1A, 4A, and FIG. 6A). However, all of the mice that were allowed to continue beyond 5 months (4 out of the 9 successful BAT transplants) reverted back to the lower baseline within a month of the hyperglycemic episode, and recovered normal glucose tolerance similar or better than their 3 month levels (FIGS. 1A, 4A, and FIG. 6A). These results are from immune-competent mice, and similar results were also seen following BAT transplants into STZ-treated Nude mice (FIGS. 2A-F). In nude mice the BAT transplants performed better, achieving euglycemia and complete normalization of glucose tolerance by 2 months post-transplant.

BAT Transplants Act Independent of Insulin.

Plasma insulin levels in BAT transplant animals remain statistically identical to those from untreated diabetic mice. Unlike non-diabetic control mice, BAT transplant mice show no insulin increase in response to a glucose challenge, a response that is comparable to the diabetic pre-transplant situation as well as untreated diabetic controls (FIG. 2A). The first phase of insulin release is missing in the transplant recipients (FIG. 2E), nor is it observed as the mice reach euglycemia following transplants. Arginine, which elicits a robust insulin response above and beyond GSIS from normal mice, also fails to stimulate an insulin response from transplant mice (FIG. 2F). To determine any possible contribution from endogenous insulin, the inventors examined pancreatic morphology by histology and insulin immunofluorescence. Histological analysis does not reveal any intact islets nor any cellular insulin immunostaining in the pancreata from BAT transplant animals (FIG. 2D). Islet morphology and cellular insulin staining is lacking in both diabetic control (FIG. 2C) and BAT transplant animals, in contrast to normal control animals (FIG. 2B). Pancreatic insulin content in the BAT transplant mice does not differ significantly from diabetic mice, both of which are >10 fold lower than normal non-diabetic control animals (Table 2).

Euglycemia Negatively Correlates with Adipose Tissue Inflammation.

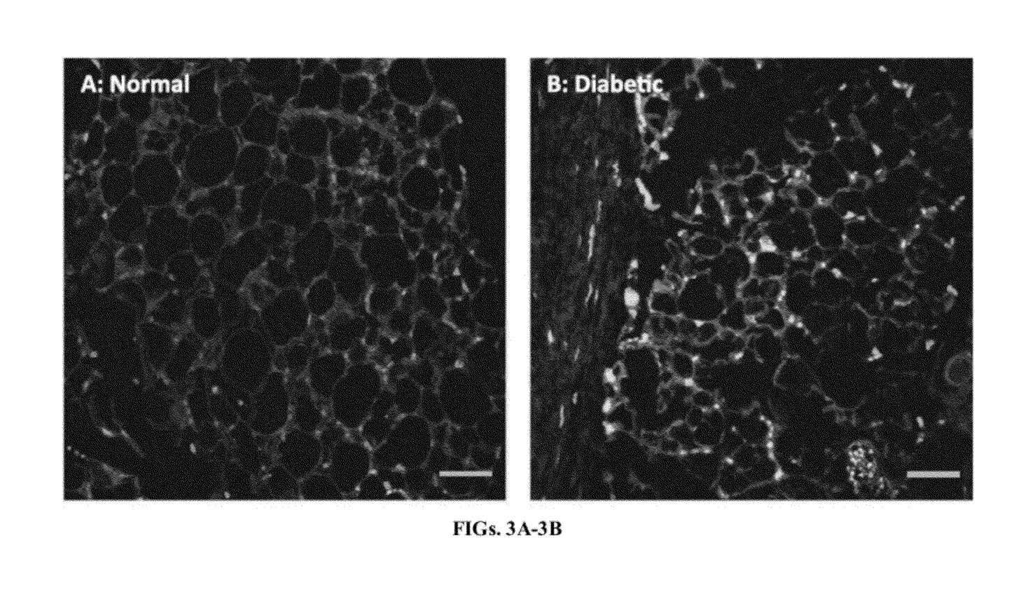

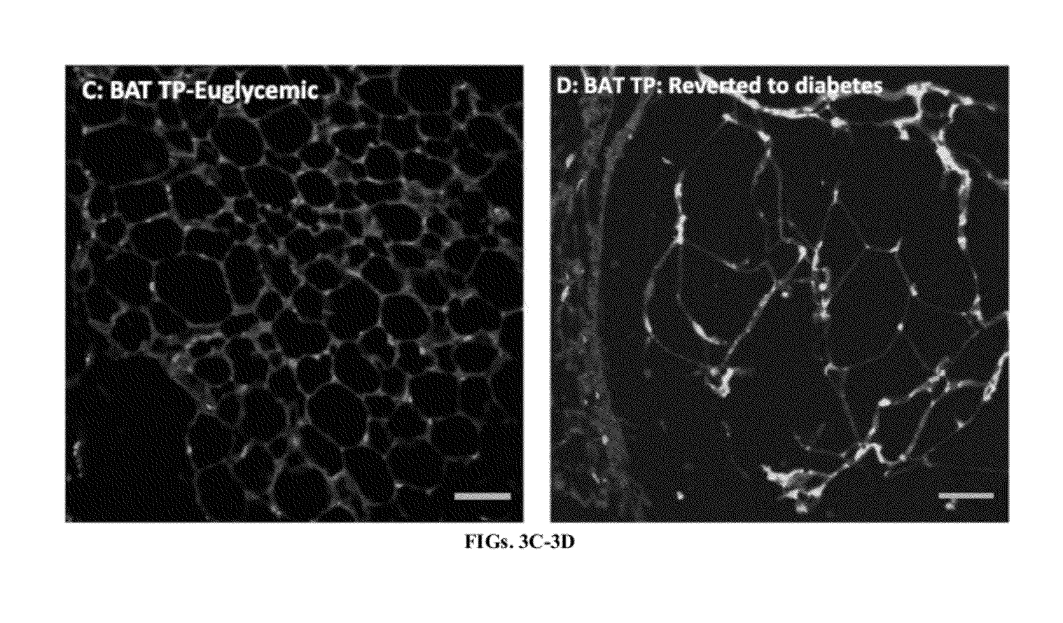

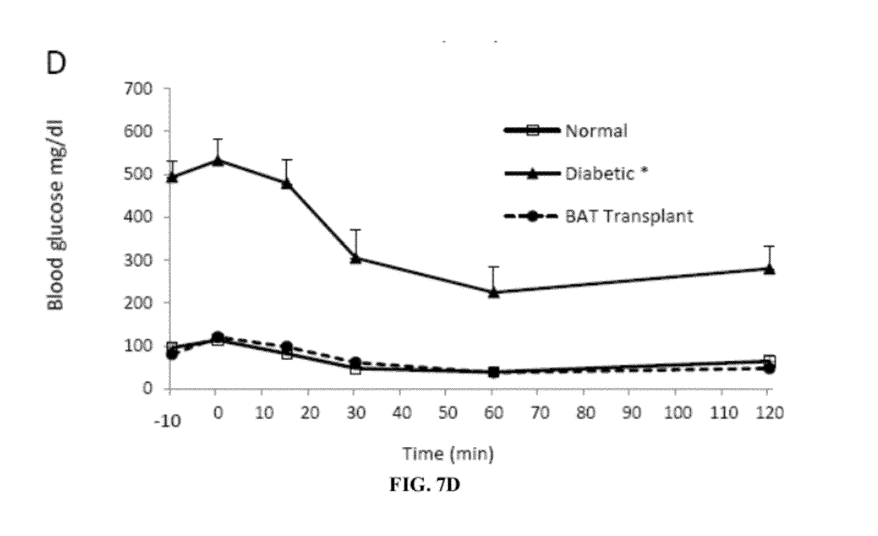

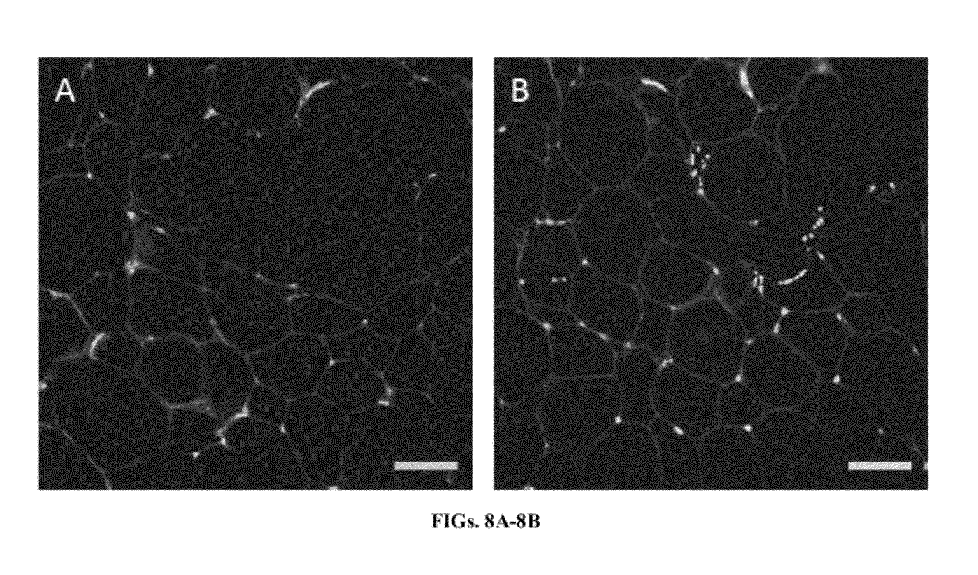

STZ-treated animals exhibit significant adipose tissue inflammation (Lin et al., 2005; Yessoufou et al., 2011, FIG. 3B). This inflammation is characterized by elevation of adipokines, such as IL-6 and TNFα. In the BAT transplant animals, there is recovery and expansion of subcutaneous white adipose tissue (WAT), which contribute to the observed weight gain. Immunofluorescence staining of WAT from the recipient's own fat bed (i.e., not taken from the transplant site) shows low levels of the pro-inflammatory cytokines IL-6 and TNFα (FIG. 3C), with adipocyte diameters of 22.68±1.52 μm. Both these parameters are similar to those seen in non-diabetic control animals (FIG. 3A), where adipocyte diameters are 22.74±1.89 μm. In contrast, adipose tissue from untreated diabetic control animals (FIG. 3B), exhibits increased IL-6 and TNFα levels, increased adipocyte diameters of 35.88±3.57 μm, and infiltration of macrophages resulting in thickening of cell membranes, all of which is consistent with a severe inflammatory phenotype. Similar inflammatory phenotypes are seen in unsuccessful BAT transplants (i.e., transplant recipients that never achieved euglycemia, FIG. 8), and successful BAT transplants that reverted to diabetes after 4 months (FIG. 3D), although these groups exhibit even larger adipocytes of 78.53±5.24 μm. Thus, inflammation appears to be directly related to loss of ability of adipose tissue to maintain glucose homeostasis.

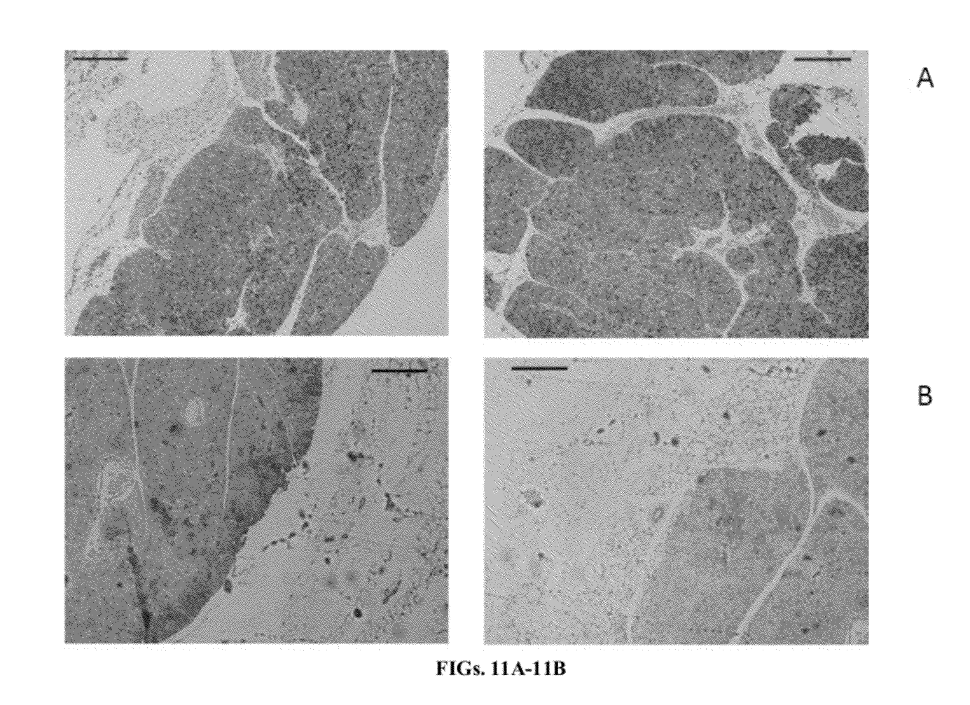

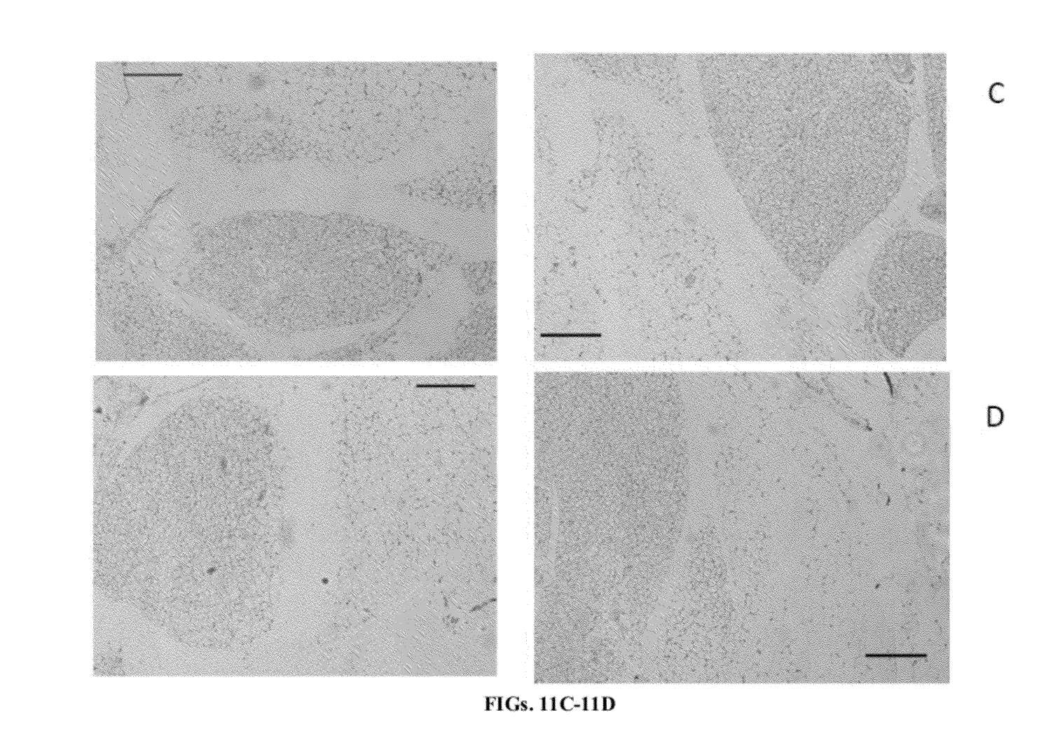

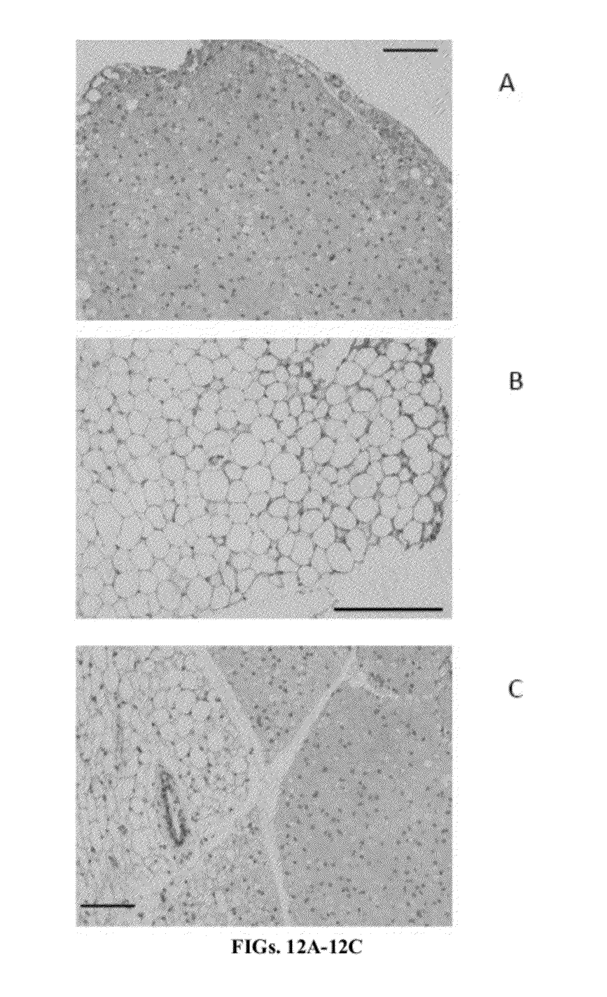

The eventual fate of the transplanted brown adipose tissue was monitored by histological examination of the transplant site at different time points. UCP-1 is observed by immunostaining at 2 months post-transplant, but not at 3 months or beyond (FIGS. 11A-D). The UCP-1 staining is localized to areas smaller than the original embryonic BAT transplant, and the level of UCP-1 staining decreases over time. While UCP-1 staining declines over time, BAT appears to expand into the WAT, as seen in the tissue morphology at 3 and 6 months (FIGS. 11C-D, FIG. 12C).

Non-Insulin Regulators of Glucose Homeostasis.

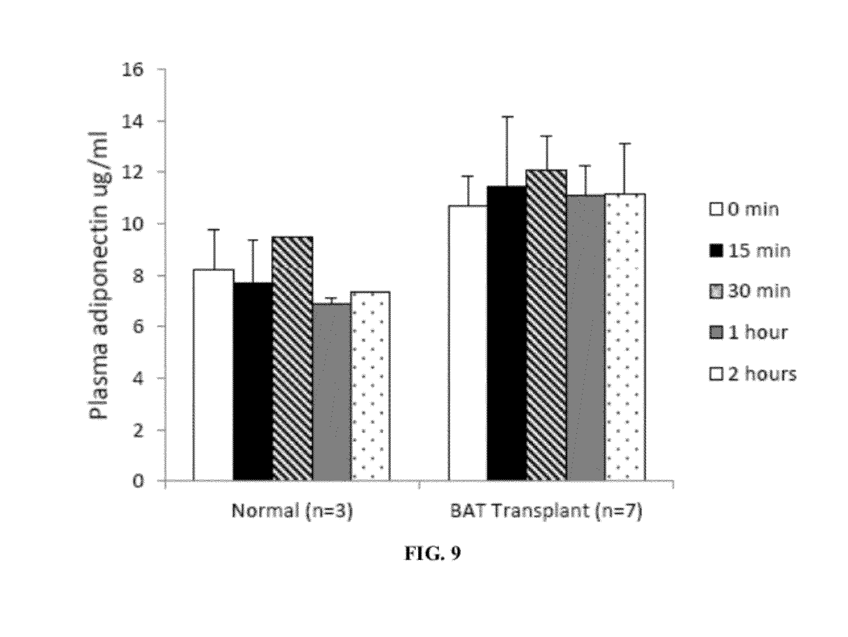

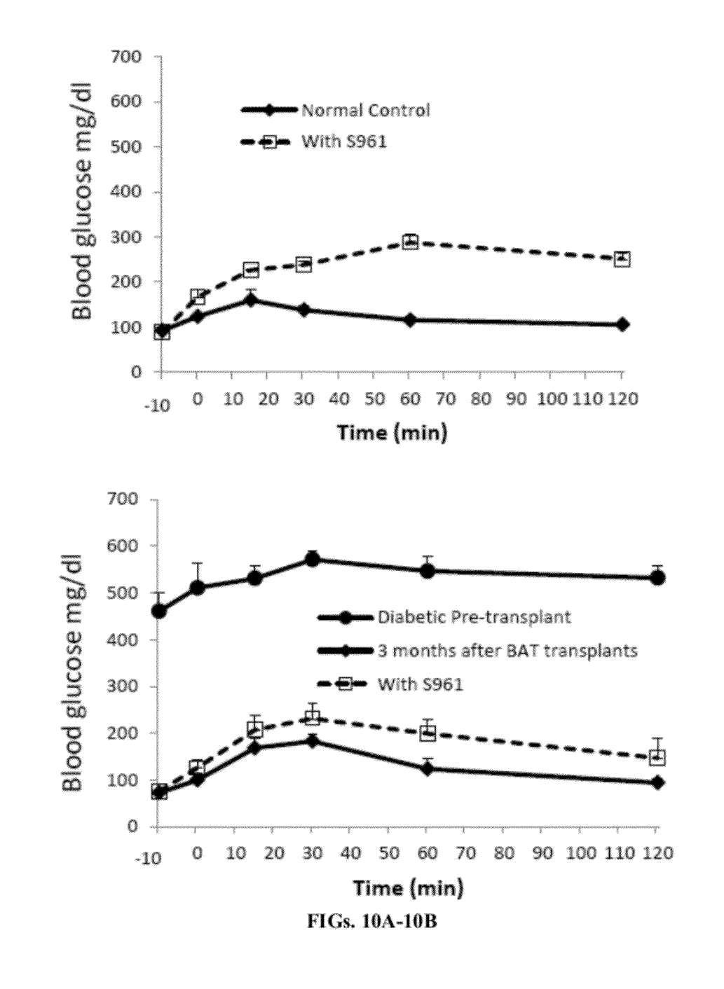

To determine the molecular mechanisms underlying the insulin-independent glucose control, the inventors measured the blood plasma levels of two candidate adipokines, adiponectin and leptin (FIGS. 4A-F). Several studies from different sources indicate the ability of adiponectin to improve cell growth and increase insulin sensitivity in peripheral tissues, particularly adipocytes (Luo et al., 2010; Fu et al., 2005; Li et al., 2009; Snehalatha et al., 2003; Kaas et al., 2010). In the BAT transplant animals, plasma adiponectin levels show considerable correlation with glucose homeostasis: low levels pre-transplant, with gradually increasing levels as glucose homeostasis improved (FIG. 4C). The adiponectin levels directly mirror the plasma glucose levels (FIG. 4A). Basal and glucose-stimulated levels of adiponectin are not significantly different, and there is no change of adiponectin levels in responses to a glucose challenge (FIG. 9). Mirroring the behavior of plasma glucose levels, plasma adiponectin levels also exhibit a marked decrease at four months corresponding with the transient hyperglycemic spike, and then returned to high levels corresponding with the recovery of euglycemia.

Plasma leptin levels show a similar time course after BAT transplant, and also exhibit a dip and recovery around 4 months post-transplant (FIG. 4D). As is seen for adiponectin, there is also a distinct correlation between improved glucose homeostasis and increased leptin levels. Leptin is reported to affect glucose homeostasis by suppressing glucagon and decreasing lipogenesis (Yu et al., 2008; Wang et al., 2010). Thus, the inventors examined glucagon levels in the BAT transplant recipients compared with other groups (FIG. 4E). Plasma glucagon levels increase in the STZ-treated animals, and decrease significantly 3 months past-transplant. This decrease in glucagon was accompanied by an elevation of plasma IGF-1 levels (FIG. 4F), which is similar to what is observed after exogenous leptin treatment (Yu et al., 2008). Notably, the glucagon levels in BAT transplant recipients were significantly lower than those in the non-diabetic control group, and IGF-1 levels were significantly higher. Thus, suppression of glucagon may be a key mechanism of glucose homeostasis that becomes even more important in the absence of insulin, as has also been reported in recent studies (Lee et al., 2011; Edgerton and Cherrington, 2011). In addition, IGF-1 may also compensate for insulin function, as evidenced by the experiments with insulin receptor inhibition described in the next section. Upon histological examination of adipose tissue sections from the transplant site at 2, 3, and 6 months, immunostaining for IGF-1 was consistently observed in the BAT, while IGF-1 staining in the expanded WAT varied from mild to none (FIGS. 12A-C).

Role of the Insulin Receptor.

In the BAT transplant model, diabetes is corrected without an increase in endogenous insulin or addition of exogenous insulin. However, the adipose tissue appears to be normal, and activation of the insulin receptor is believed to be critical for adipose tissue health and survival (Laviola et al., 2006). To explore the possible interactions between the elevated adipokines and the insulin receptor, the inventors monitored glucose tolerance in the presence of S961, a competitive inhibitor of the insulin receptor (Schäffer et al., 2008). In non-diabetic control mice, S961 causes an impairment of glucose tolerance (FIG. 5A). In the presence of S961, blood glucose levels increase after the addition of a glucose bolus, but do not show a significant return to basal levels even after two hours. In animals with successful BAT transplants, S961 causes a similar impairment of glucose tolerance (FIG. 5B), indicating that the insulin receptor plays a role in the insulin-independent regulation of glucose homeostasis. STZ-treated Nude mice also exhibit similar glucose tolerance test results in the presence of S961 (FIGS. 11A-D).

TABLE 1
Average body weight, and content of subcutaneous adipose tissue
collected from dorsal body surface at post mortem from representative
subjects from each group

| | Body wt | Adipose tissue | Adipose tissue |
|---|---|---|---|
| Group | (grams) | (grams) | (% body wt) |
| Normal non-diabetic | 27.25 ± 0.94 | 0.15 ± 0.0075 | 0.56 ± 0.032 |
| (n = 4) | | | |
| Untreated diabetic | 23.6 ± 0.88 | 0.08 ± 0.009 | 0.34 ± 0.028 |
| (n = 5) | | | |
| Successful BAT | 31.5 ± 0.51* | 0.33 ± 0.027** | 1.05 ± 0.09 |
| transplants (n = 5) | | | |
| Failed BAT | 27.8 ± 1.76 | 0.14 ± 0.015 | 0.5 ± 0.022 |
| Transplants (n = 3) | | | |

*= p < 0.005, when compared with untreated diabetic group
**= p < 0.001 when compared with untreated diabetic group, and p < 0.05 when compared with other groups

TABLE 2
Average pancreatic insulin content at post mortem from each group

| | Pancreatic Insulin Content |
|---|---|
| Group | (ng/mg tissue) |
| Normal non-diabetic (n = 7)* | 146.65 ± 20.19 |
| Untreated diabetic (n = 5) | 12.01 ± 5.3 |
| Successful BAT transplants (n = 6) | 15.33 ± 2.43 |
| Failed BAT transplants (n = 5) | 9.2 ± 5.59 |

*= p < 0.001, when normal non-diabetic group is compared with all other groups. No significant difference between the other groups.

### Example 3

### Discussion

Here, the inventors have shown that regeneration of healthy adipose tissue can reverse clinical diabetes and re-establish glucose homeostasis with no detectable contribution from insulin (FIGS. 1-2). The correction of diabetes phenotypes by brown adipose tissue transplants in STZ-treated mice can persist for over six months, without immunosuppression or hormone supplements (FIG. 1). These data suggest that these BAT transplants achieve chronic regulation of glucose through a steady-state elevation of alternative hormones, such as adiponectin, leptin and IGF-1 (FIG. 4). This regulation correlates with the maintenance of healthy and non-inflamed adipose tissue, as demonstrated by the inflammatory changes in adipose tissue associated with the diabetic state (FIG. 3). Although insulin is not required, activation of the insulin receptor still appears to play a physiological role since its blockade leads to impaired glucose tolerance in the transplant recipients (FIG. 5).

The lack of insulin response is confirmed by consistently low plasma insulin levels in the transplant recipients (FIG. 1A) and a lack of insulin content (Table 2) in the pancreas post mortem. Previous studies have reported recovery of beta cell mass and function in STZ-treated mice (Grossman et al., 2010; Yin et al., 2006). However, plasma insulin levels and insulin response to a glucose or arginine challenge remained consistently low, comparable to untreated diabetic controls and in stark contrast to normal controls (FIGS. 2A, 2E and 2F). Further, histological examination from successful BAT transplant recipients shows no insulin immuno-reactivity in their pancreata (FIG. 2D). Thus, recovery of islet mass and function does not play a significant role in the glucose homeostasis of BAT transplant animals.

Although insulin is not required, activation of the insulin receptor does appear to be involved (FIG. 5). This involvement is in contrast to the proposed mechanisms underlying leptin treatment that focus solely on its role in the inhibition of glucagon action (Wang et al., 2010). Molecules that activate the insulin receptor may include IGF-1, whose levels are significantly elevated in BAT transplant mice compared to both normal and diabetic groups (FIG. 3F). The inventors also find that IGF-1 is expressed in the transplanted BAT (FIG. 12). Several studies have reported the beneficial effects of IGF-1 in improving glucose metabolism and glucose tolerance (LeRoith and Yakar, 2007; Zenobi et al., 1992; Rao et al., 2010; Zenobi et al., 1994; Dunger et al., 2004). Administration of recombinant IGF-1 improves glucose and lipid metabolism in a range of conditions including type 2 diabetes, obesity and type 1 diabetes (LeRoith and Yakar, 2007; Zenobi et al., 1992; Rao et al., 2010; Zenobi et al., 1994), whereas low IGF-1 levels are associated with impaired glucose tolerance (Dunger et al., 2004). The inventors' histological studies show consistent presence of IGF-1 in the transplanted BAT, with a lesser degree of immuonstaining in the expanded WAT. While part of the IGF-1 may arise from the transplant site, it is likely that elevated IGF-1 levels in plasma are generated from elsewhere, possibly stimulated by increased leptin produced from new WAT. Recent studies show the ability of leptin to stimulate the expression of IGF-1 in various tissues (Dumond et al., 2003; Soliman et al., 2000; Hamrick and Ferrari, 2008).

A central role for adipose tissue hormones in glucose homeostasis is increasingly supported in the literature. Certain adipokines, such as resistin, retinol binding protein and TNFα, have long been recognized for their ability to exacerbate hyperglycemia (Tamori et al., 2006; Ondrak and Hackney, 2010; Choi et al., 2008; Schreyer et al., 1998)). However, several more recent studies show that individual adipokines can correct diabetes independent of insulin (Hu et al., 2007; Fukushima et al., 2007; Yu et al., 2008; Wang et al., 2010). In particular, leptin has been shown to restore euglycemia in STZ-treated animals, putatively though its role in reducing hyperglucagonemia (Yu et al., 2008; Wang et al., 2010; Lee et al., 2011; Edgerton and Cherrington, 2011). Adiponectin gene therapy has also been shown to ameliorate both type 1 and type 2 diabetes (Hu et al., 2007; Fukushima et al., 2007), an effect that is proposed to act through increased insulin receptor sensitivity (Luo et al., 2010; Fu et al., 2005). Previous studies further show that adiponectin and leptin each bind to specific receptors while also influencing the actions of other hormones through indirect mechanisms (Wozniak et al., 2009; Kadowaki and Yamauchi, 2005). These data show fluctuations of plasma adiponectin and leptin levels correlate directly with glucose homeostasis in the absence of insulin, along with elevated levels of IGF-1 and reduced levels of plasma glucagon. Taken together, these data suggest a model where glucose homeostasis is regulated by the combination of adipokine activation of insulin receptor signaling (or an equivalent downstream pathway), possible replacement of insulin function with IGF-1, and leptin-mediated reduction of hyperglucagonemia. Another possibility is that adiponectin improved the overall health of peripheral tissues and increased sensitivity to very low levels of insulin which normally would not have been sufficient to maintain euglycemia. Indeed, since approximately one third of the transplants failed (i.e. the replenished adipose tissue failed to reverse diabetes and showed inflammatory changes post-mortem), it is possible there is still some requirement for sub-detectable levels of insulin. If such a requirement exists, it might be present only at the outset of the BAT transplant. In any case, such a need may be satisfied clinically with the residual insulin levels seen in recently diagnosed T1D patients. Alternatively, this need could be met by provision of sub-physiological levels of insulin (which would not produce the side effects associated with traditional insulin therapy) or by other methods such as administration of hormone sensitive lipase levels sufficient to maintain adipose tissue.

While the possibility of insulin-independent glucose regulation by adipokines is established in previous studies, development of therapies based on these effects would still be hampered by limitations of compound delivery. Such therapies would likely require persistent injections or infusion pumps which necessitate careful dosage calculations to avoid deleterious effects. Successful treatments could eventually rely on gene therapy, but this approach is still unproven for clinical use. These data demonstrate the ability of whole adipose tissue to compensate for loss of the insulin secreting β-cells, providing a simpler therapeutic approach. Transplantation of embryonic brown adipose tissue at appropriate gestational ages stimulates regeneration of recipients' subcutaneous white adipose tissue, which in turn secretes a combination of adipokines that bring about euglycemia. This approach does not require any further intervention, and thus could constitute a legitimate cure. Therapies based on this approach would provide a safe and convenient method to utilize the known effects of adipokines for T1D treatment without the complications associated with regular delivery of exogenous compounds.

### Example 4

### Clinical Protocol

One surgical procedure is to transplant the BAT into the subcutaneous space. This procedure requires a small (˜4 cm) incision, and donor tissue is introduced into the pocket with forceps. The incision is closed by gentle pressure with hemostats and sutured. Approximately 1 mg (or ˜10⁷ cells) of BAT transplant tissue is required per 50 pounds of recipient body weight. Surgeries are performed under local anesthesia with post-operative analgesia as needed.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VI. REFERENCES

The following references, to the extent that they provide exemplary procedural or other details supplementary to those set forth herein, are specifically incorporated herein by reference.
U.S. Patent Publn. 2010/0028310
U.S. Patent Publn. 2011/0144009
U.S. Patent Publn. 2010/0150885
Choi et al., J Clin. Endocrinot Metab., 93:3142-3148, 2008.
Dumond et al., Arthritis Rheum., 48(11):3118-3129, 2003.
Dunger et al., Horm. Res., 62(Suppl 1):101-107, 2004.
Edgerton and Cherrington, Diabetes, 60:377-380, 2011.
Fu et al., J. Lipid Res., 46:1369-1379, 2005.
Fukushima et al., J. Gene Med., 9:976-85, 2007.
Grossman et al., PLoS One, 5(1):e8749, 2010.
Hamrick and Ferrari, Osteoporos Int., 19(7):905-912, 2008.
Hu et al., Acta Biochim. Biophys. Sin. (Shanghai), 39:131-136, 2007.
Kaas et al., Clin. Exp. Immunol., 161:444-452, 2010.
Kadowaki and Yamauchi, Endocrine Rev., 26:439-451, 2005.
Laviola et al., Arch. Physiol. Biochem., 112:82-88, 2006.
Lee et al., Diabetes, 60:391-397, 2011.
LeRoith and Yakar, Nat. Clin. Pract. Endocrinol. Metab., 3(3):302-310, 2007.
Li et al., JAMA, 302:179-188, 2009.
Lin et al., J. Biol. Chem., 280:4617-4626, 2005.
Luo et al., Diabetes, 59:791-799, 2010.
Ondrak and Hackney, Med. Sport Sci., 55:32-42, 2010.
PCT Appln. WO 2010/022262
Rao et al., J. Clin. Endocrinot Metab., 95(9):4361-4366, 2010.
Schäffer et al., Biochem. Biophys. Res. Commun., 376:380-383, 2008.
Schreyer et al., J. Clin. Invest., 102:402-411, 1998.
Seale et al., J. Clin. Invest., 121(1):96-105. 2011.
Serra-Renom et al., Plast. Reconstr. Surg. 125 (1):12-8 (2010).
Snehalatha et al., Diabetes Care, 26:3226-3229, 2003.
Soliman et al., Metabolism, 49(7):819-825, 2000.
Tamori et al., Nature Med., 12:30-31, 2006.
Tran and Kahn, Nat Rev Endocrinol. 6:195-213, 2010.
Wang et al., Proc. Natl. Acad. Sci. USA, 107:4813-4819, 2010.
Wozniak et al., Dig. Dis. Sci., 54:1847-1856, 2009.
Yessoufou et al., J. Obes., pii:654967, 2011.
Yin et al., Diabetes, 55(12):3256-3263, 2006.
Yu et al., Proc. Natl. Acad. Sci. U.S.A. 105:14070-14075, 2008.
Zenobi et al., Eur. J. Endocrinol., 131(3):251-257, 1994.
Zenobi et al., J. Clin. Invest., 90(6):2234-2241, 1992.

1. A method of treating a subject having diabetes requiring insulin treatment comprising providing to said subject brown adipose tissue.

2. The method of claim 1, wherein said diabetes is other than lipoatrophic diabetes.

3. The method of claim 1, wherein said subject is a human.

4. The method of claim 1, wherein said subject is a non-human animal.

5. The method of claim 1, wherein said diabetes is type 1 diabetes, type 2 diabetes, neonatal diabetes, or MODY diabetes.

6. The method of claim 1, wherein said subject suffers from autoimmune type 1 diabetes.

7. The method of claim 1, wherein said subject suffers from toxin-induced type 1 diabetes, chemically-induced type 1 diabetes, and genetically-induced type 1 diabetes.

8. The method of claim 1, wherein providing comprises surgically transplating a brown adipose tissue sample into said subject.

9. (canceled)

10. The method of claim 1, further comprising administering to said subject one or more of insulin, amylin, leptin, a sulfonylurea or a GLP-1 analog.

11-14. (canceled)

15. The method of claim 1, wherein no exogenous insulin is provided.

16. The method of claim 1, wherein said subject is essentially devoid of endogenous insulin.

17. The method of claim 1, wherein said providing achieves a venous or capillary fasting blood glucose (FBG) levels of less than 200 mg/dl, less than 175 mg/dl, less than 150 mg/dl, less than 140 mg/dl, less than 130 mg/dl, less than 126 mg/dl, less than 120 mg/dl, or less than 115 mg/dl, less than 110 mg/dl, or less than 100 mg/dl.

18. The method of claim 1, wherein BAT is produced from donor tissue.

19. The method of claim 1, wherein BAT is produced from tissue culture.

20. The method of claim 19, wherein BAT is produced from a cell line, an embryonic stem cell, an induced pluripoent stem cell, a hematopoietic stem cell or an adipose stem cell.

21. A method of restoring normoglycemia in a subject diagnosed with diabetes requiring insulin treatment comprising providing to said subject brown adipose tissue.

22. The method of claim 21, further comprising administering to said subject one or more of insulin, amylin, leptin, a sulfonylurea or a GLP-1 analog.

23. The method of claim 22, wherein said subject receives no more than about 10% of a normal daily dosage of insulin supplementation.

24. The method of claim 21, wherein said subject receives no insulin supplementation.

25. (canceled)

26. A method of reducing, suppressing, attenuating, or inhibiting hyperglucogonemia or a condition associated with hyperglucogonemia in a subject having diabetes requiring insulin treatment comprising providing to said subject brown adipose tissue.

27. The method of claim 26, further comprising administering to said subject one or more of insulin, amylin, leptin, a sulfonylurea or a GLP-1 analog.

28. The method of claim 27, wherein said subject receives no more than about 10% of a normal daily dosage of insulin supplementation.

29. The method of claim 26, wherein said subject receives no insulin supplementation.

30. (canceled)

31. A method of increasing adiponectin, visfatin, and/or leptin in a subject having type I diabetes comprising providing to said subject brown adipose tissue.

32. The method of claim 31, further comprising administering to said subject one or more of insulin, amylin, leptin, a sulfonylurea or a GLP-1 analog.

33. The method of claim 32, wherein said subject receives no more than about 10% of a normal daily dosage of insulin supplementation.

34. The method of claim 31, wherein said subject receives no insulin supplementation.

35. (canceled)

36. A method of treating pre-diabetic metabolic syndrome in a subject comprising providing to said subject brown adipose tissue.


SEQUENCE LISTING (1 sequence)

SEQ ID NO: 1 - protein, 37 aa, Homo sapiens [sequence omitted]